(19) United States
(12) Patent Application Publication
(10) Pub. No.: US 2023/0406924 A1
(43) Pub. Date: Dec. 21, 2023

(54) USE OF ANTI-TREM1 NEUTRALIZING ANTIBODIES FOR THE TREATMENT OF MOTOR NEURON NEURODEGENERATIVE DISORDERS

(71) Applicant: UCB BIOPHARMA SRL, Brussels (BE)

(72) Inventors: Irena KADIU, Brussels (BE); Julien GASSER, Brussels (BE); James Martin KEANEY, Brussels (BE); Anastasios SPILIOTOPOULOS, Slough, Berkshire (GB); Jonathan Mark PHILLIPS, Slough Berkshire (GB)

(21) Appl. No.: 18/251,006

(22) PCT Filed: Nov. 2, 2020
(86) PCT No.: PCT/EP2020/080672

§ 371 Date: Apr. 28, 2023

(51) Int. Cl. C07K 16/28; A61P 25/28

(52) U.S. Cl. CPC C07K 16/2803; A61P 25/28; C07K 2317/92

(57) ABSTRACT

The present invention provides antibodies or antigen-binding fragment thereof that bind and neutralize TREM1 for use in the treatment of a motor neuron degenerative disorders, in particular amyotrophic lateral sclerosis.

The present invention relates to anti-TREM1 antibodies for use in the treatment of motor neuron degenerative disorders, and more particularly, for the treatment of amyotrophic lateral sclerosis (ALS).

### BACKGROUND

Amyotrophic lateral sclerosis (ALS) is a multifactorial neurodegenerative disease caused by genetic and non-inheritable factors leading to motoneuron degeneration in the spinal cord, brain stem and primary motor cortex (Al-Chalabi and Hardiman, 2013). 90% of the ALS cases are sporadic (sALS), while 5/6-20% report a familial history of the disease (fALS) (Al-Chalabi et al., 2017).

The large majority of familial ALS cases are due to genetic mutations in the Superoxide dismutase 1 gene (SOD1) and repeat nucleotide expansions in the gene encoding C9ORF72 (around 40-50% of familial ALS and ˜10% of sporadic ALS) (Philips et al, 2015). The most commonly used transgenic mouse model of ALS, the SOD1 mouse, recapitulates many symptoms of the human ALS pathology. SOD1 mouse models have been extensively studied in basic and translational research with the purpose of understanding the mechanism of ALS and possible ways of treating this condition.

Sporadic and familial forms of ALS share most neuropathological features. Similar biological pathways are affected in both (Butovsky et al, 2012; Lincencum et al, 2010). At the same time clinical presentation is heterogeneous regarding age, site of disease onset, rate of disease progression and survival (Beers, D. R. et. al. 2019). ALS is a non-cell autonomous disease. Processes leading to motor neuron death are multifactorial and reflect complex interactions between genetic and environmental factors. Evidence from clinical studies suggests that a dysregulated immune response contributes to this heterogeneity.

Neuroinflammation (aberrant microglia and peripheral immune activation) is a common denominator and converging point of pathologic mechanisms driving genetic and sporadic forms of ALS. It is not entirely clear whether immune activation is involved in disease initiation, although epidemiological studies have shown autoimmune disease, including asthma, celiac disease, ulcerative colitis and others precede ALS (Turner et al., 2013). Additionally studies in pre-clinical models of disease suggest that modulation of microglia significantly impacts the rate of disease progression (Harms et al., 2014; Boille et. al., 2006).

Triggering receptors expressed on myeloid cells (TREM) are receptors that include immune-activating and -inhibitory isoforms encoded by a MHC gene cluster mapping to human chromosome 6P21 and mouse chromosome 17. TREMs are members of the immunoglobulin (Ig) superfamily primarily expressed in cells of the myeloid lineage including monocytes, neutrophils, and dendritic cells in the periphery and microglia in the central nervous system (CNS). Triggering receptor expressed on myeloid cells-1 (TREM1) is the first member of the TREM family to be identified and it has limited homology with other receptors of the Ig superfamily. TREM1 is transmembrane glycoprotein with a single Ig-like domain, a transmembrane region with a (+) charged lysine residue interacting with a negatively charged aspartic acid on its signaling partner DAP12 and a short cytoplasmic tail that lacks any signaling domains (Colona, 2003). TREM1 activation either through interactions with its proposed natural ligands such as peptidoglycan recognition protein 1 (PGRP1), high mobility group B1 (HMGB1), soluble CD177, heat shock protein 70 (HSP70) has been proposed to induce formation of an “head-to-tail’ TREM1 homodimer. Crosslinking triggers the phosphorylation of the immune receptor tyrosine-based activating motif (ITAM) on the recruited DAP12, which enables signaling and function by providing with a docking site for spleen tyrosine kinase (SYK) and its downstream signaling partners including zeta-chain-associated protein kinase 70 (ZAP70), casitas b-lineage lymphoma (Cbl), son of sevenless (SOS) and growth factor receptor binding protein 2 (GRB2). These interactions trigger downstream signal transduction through phosphatidylinositol 3-kinase (PI3K), phospholipase-C-γ 2 (PLC-γ2) and the ERK pathways. These events are followed by mobilization of calcium mobilization, activation of transcription factors including ETS-containing protein (ELK1), nuclear factor of activated T-cells (NFAT), AP1, c-fos, c-Jun and NF-κB. These pathways triggered either by interactions with its ligand or through TREM1 interaction with various members of Toll-like receptor and NOD-like receptors (NLR) result downstream in the release of pro-inflammatory cytokines (MCP1, IL-8, MCSF, IL6, TNFα, IL-β etc.), increase in costimulatory molecules in monocytes and dendritic cells and degranulation in neutrophils (Buchon et al, 2000).

US 2018/0318379 discloses that any an antisense agent, an RNAi agent, a genome editing agent, antibody or peptide that inhibits TREM1 activity and/or expression could be used for treating a subject having an acute or chronic central nervous system disorder.

U.S. Pat. No. 9,000,127 provides anti-TREM1 antibodies that disrupt the interaction of TREM1 with its ligand. The disclosed antibodies are provided for the treatment of individuals with an inflammatory disease, such as rheumatoid arthritis and inflammatory bowel disease.

WO 2017/152102 discloses antibodies that bind to a TREM1 protein and modulate or enhance one or more TREM1 activities.

While considerable research has been done into understanding the mechanisms of ALS, there remains a significant interest in and need for additional or alternative therapies for treating, preventing and/or delaying the onset and/or development of ALS. The present invention addresses that need.

The present invention for the first time demonstrates that antibodies binding and neutralizing TREM1 are effective in the treatment of motor neuron degeneration conditions, more specifically, ALS. The invention for the first demonstrates that anti-TREM1 antibodies attenuate brain and spinal cord inflammation by reducing microglia neuronal uptake and microglia migratory activities in vivo.

### SUMMARY OF THE INVENTION

The present invention demonstrates the role of TREM1 is a key potentiator of microglia maladaptive neurotoxic responses in the context of neuron degenerative disorders such as ALS. Specifically, TREM1 modulation reduces microglia neuronal uptake, pro-inflammatory cytokine release and microglia/peripheral immune migratory activities in vitro, ex vivo and in vivo models of ALS and attenuates brain and spinal cord inflammation in a SOD1G93A mouse model of ALS. The present invention for the first time demonstrates that systemically injecting anti-TREM1 antibody that neutralizes TREM1 provides sufficient levels of such in brain and spinal cord tissues to reduce the ALS disease phenotype and achieve therapeutic effect.

The present invention provides a method of treating a motor neuron degenerative disorder in a subject in need thereof, the method comprising administering to the subject an anti-TREM1 antibody or antigen-binding fragment thereof.

The present invention also provides an anti-TREM1 antibody or antigen-binding fragment thereof for use in the treatment of a motor neuron degenerative disorder.

The present invention also provides use of an anti-TREM1 antibody or antigen-binding fragment thereof for the manufacture of a medicament for the treatment of a motor neuron degenerative disorder.

More specifically the motor neuron degenerative disorder is amyotrophic lateral sclerosis.

### BRIEF DESCRIPTION OF THE DRAWINGS

The present invention is described below by reference to the following drawings, in which:

FIG. 1 shows that uptake of zymosan particles was reduced in TREM1−/− microglia relative to WT microglia. As shown in FIG. 1B, this reduction in the rate of phagocytosis in TREM1−/− microglia was statistically significant (error bars±SEM; 30 min timepoint; ****p<0.0001; Student's t-test).

FIG. 2 shows that microglial migration into the wound area at 24 hours post-wound was lower in TREM1−/− microglia relative to WT microglia. Black lines indicate the initial wound boundaries. As shown in FIG. 2B, this reduction in the migratory capacity of TREM1−/− microglia was statistically significant (16 hour and 24 h timepoints; error bars±SEM; ****p<0.0001; Student's t-test).

FIG. 3 shows that following LPS stimulation, levels of MCP-1 were significantly lower in supernatants collected from TREM1−/− microglia relative to WT microglia (error bars±SEM; **p<0.01; Student's t-test).

FIG. 4 shows that BV2 migration into the wound area at 24 hours post-wound was lower in anti-TREM1-treated microglia relative to isotype antibody- or vehicle-treated microglia. Black lines indicate the initial wound boundaries. As shown in FIG. 4B, this reduction in the migratory capacity of anti-TREM1-treated BV2 microglia was statistically significant (24 h timepoint; error bars±SEM; *p<0.05, **p<0.01; one-way ANOVA followed by Tukey's multiple comparisons test).

FIG. 5 shows that treatment of MDMs with PGN-BS alone or PGN-BS+PGLYRP1 increased release of TNF-α, IL-1β, IL-6 and IL-8 from MDMs from 3 different donors relative to PGLYRP1 alone or untreated controls (error bars±SEM; ****p<0.0001; two-way ANOVA followed by Tukey's multiple comparisons test of the global means of all donors). For 3 out of 3 donors, IL-1β levels were higher following PGN-BS+PGLYRP1 treatment relative to PGN-BS alone (error bars±SEM; ***P<0.001; two-way ANOVA followed by Tukey's multiple comparisons test of the global means of all donors). For 2 out of 3 donors, TNF-α and IL-6 levels were higher following PGN-BS+PGLYRP1 treatment relative to PGN-BS alone (error bars±SEM;***p<0.001; two-way ANOVA followed by Tukey's multiple comparisons test of the global means of all donors). IL-8 levels were not significantly different between PGN-BS and PGN-BS+PGLYRP1 treatments in all 3 donors.

FIG. 6 shows that uptake of zymosan particles and the number of Iba1+ phagocytic cells was reduced in TREM1−/− microglia relative to WT microglia. As shown in (B) and (C), this reduction in the number of phagocytosed bioparticles and of Iba1+ phagocytic cells were statistically significant (error bars±SEM; n=5 for TREM1−/− and n=6 for WT; n=23 total brain sections per genotype for TREM1−/− and 14 for WT; *p<0.05; Student's t-test).

FIG. 7 shows that uptake of synaptosomes was significantly reduced in the TREM1−/− microglia relative to WT microglia (error bars±SEM; n=3 mice per genotype, n=12 brain sections per genotype, *p<0.05; Student's t-test).

FIG. 8 shows that microglia from TREM1−/− mice also showed a striking change in their morphology. As shown in FIGS. 8B and 8C, this modification in morphology is reflected by significantly longer and more ramified processes in TREM1−/− microglia compared to WT controls (error bars f SEM; n=5 mice for TREM1−/− and 6 for WT; n=28 brain sections for TREM1−/− and 14 for WT; *p<0.05; Student's t-test).

FIG. 9 shows that anti-TREM1-treated SOD1-G93A mice showed decreased microgliosis compared to isotype-treated SOD1-G93A controls. As shown in (B), microglia from anti-TREM1-treated SOD1-G93A mice displayed reduced phagocytic uptake (microglial efficiency) compared to isotype-treated controls. There were also reduced total numbers of phagocytic microglia (microglial abundance) in anti-TREM1-treated SOD1-G93A mice. FIG. 9C shows the ventral hom region from the images in FIG. 9A. (n=5 SOD1 mice/group; Mean t SEM one-way ANOVA; *p<0.05; **p<0.01).

FIG. 10 shows that treatment of SOD1-G93A mice with a TREM1 antibody reduced levels of costimulatory molecules (CD40, CD80, CD86) and other activation markers (CD68, CSFR1) compared to isotype-treated SOD1-G93A controls (error bars±SEM; n=6 mice per treatment; **p<0.01, ***p<0.001; ****p<0.0001; Student's t-test with false discovery rate (FDR) approach). As shown in FIG. 10B, a number of costimulatory molecules and other activation markers were significantly reduced in anti-TREM1-treated SOD1-G93A mice compared to isotype-treated SOD1-G93A controls (arrows represent significant changes in anti-TREM1-treated SOD1-G93A mice).

FIG. 11 shows that t-Distributed Stochastic Neighbor Imbedding (tSNE)-analyzed and averaged data showed that 21.57% and 28.80% of all immune cells in the brain and spleen respectively were positive for the anti-TREM1 antibody in anti-TREM1-treated SOD1-G93A mice.

FIG. 12 shows (A) a 3D representation of mouse and human TREM1 with the MAB1187 epitope highlighted on the mouse TREM1 structure (left) and the human TREM1 (middle). The PGLYRP1 epitope on the human TREM1 is also shown (structure on the right). (B). Human and mouse TREM1 sequence alignment with the epitope of MAB1187 (top and middle) and PGLYRP1 (bottom) highlighted and underlined.

### DETAILED DESCRIPTION OF THE INVENTION

### Definitions

The term “antibody” as used herein generally relates to intact (whole) antibodies i.e. comprising the elements of two full length heavy chains and light chains. The antibody may comprise further additional binding domains for example as per the molecule DVD-Ig as disclosed in WO 2007/024715, or the so-called (FabFv)₂Fc described in WO2011/030107. Thus antibody as employed herein includes bi, tri or tetra-valent full length antibodies. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

As used herein the term “antigen-binding fragments” or “antigen-binding fragment” may include a conventional antigen-binding fragment structure, e.g., a Fab fragment, modified Fab, Fab′, or a F(ab′)2 fragment. An antibody can be cleaved into fragments by enzymes, such as, e.g., papain (to produce two Fab fragments and an Fc fragment) and pepsin (to produce a F(ab′)2 fragment and a pFc′ fragment). The antigen-binding fragment may also comprise a non-conventional structure (i.e., comprise antigen-binding portions of an antibody in an alternative format, which include polypeptides that mimic antigen-binding fragment activity by retaining antigen-binding capacity). In this regard, antigen-binding fragment includes domain antibodies or nanobodies, e.g., VH, VL, VHH, and VNAR-based structures, single chain antibodies (scFv), peptibody or peptide-Fc fusion, as well as di- and multimeric antibody-like molecules like dia-, tria- and tetra-bodies, or minibodies (miniAbs) that comprise different formats consisting of scFvs linked to oligomerization domains. Examples of multi-specific antigen-binding fragments include Fab-Fv, Fab-dsFv, Fab-Fv-Fv, Fab-scFv-scFv, Fab-Fv-Fc and Fab-dsFv-PEG fragments described in International Patent Application Publication Nos. WO2009040562, WO2010035012, WO2011/08609, WO2011/030107 and WO2011/061492, respectively, all of which are hereby incorporated by reference with respect to their discussion of antigen-binding moieties. Fab or Fab′ can be conjugated to a PEG molecule or human serum albumin. A further example of multi-specific antigen-binding fragments include VHH fragments linked in series. An alternative antigen-binding fragment comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antigen-binding fragments are described in International Patent Application Publication No, WO2015/197772, which is hereby incorporated by reference in its entirety and particularly with respect to the discussion of antigen-binding fragments. Antigen-binding fragments and methods of producing them are well known in the art, see for example Verma et al., 1998, Journal of Immunological Methods, 216, 165-181; Adair and Lawson, 2005. Therapeutic antibodies. Drug Design Reviews—Online 2(3):209-217. Examples of multi-specific antibodies or antigen-binding fragments thereof, which also are contemplated for use in the context of the disclosure, include bi, tri or tetra-valent antibodies, Bis-scFv, diabodies, triabodies, tetrabodies, bibodies and tribodies (see for example Holliger and Hudson, 2005, Nature Biotech 23(9): 1126-1136; Schoonjans et al. 2001, Biomolecular Engineering, 17(6), 193-202).

The term “chimeric antibody” or functional chimeric antigen-binding fragment is defined herein as an antibody molecule which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit, monkey or hamster.

As used herein, the term “humanized antibody molecule” or “humanized antibody” refers to an antibody molecule wherein the heavy and/or light chain contains one or more CDRs (including, if desired, one or more modified CDRs) from a donor antibody (e.g. a non-human antibody such as a murine monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In one embodiment only, the specificity determining residues from one or more of the CDRs described herein above are transferred to the human antibody framework. In another embodiment only the specificity determining residues from each of the CDRs described herein above are transferred to the human antibody framework.

Humanized antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanized antibodies may optionally further comprise one or more framework residues derived from the non-human species from which the CDRs were derived. The latter are often referred to as donor residues.

The terms “IgG” or “IgG immunoglobulin” or “immunoglobulin G” or “IgG antibody” as used herein are related to a polypeptide belonging to the class of antibodies that are substantially encoded by immunoglobulin gamma gene. More particular IgG comprises the subclasses or isotypes IgG1, IgG2, IgG3, and IgG4. IgG antibodies are multidomain tetrameric proteins composed of two heavy chains and two light chains. The IgG heavy chain is composed of four immunoglobulin domains linked from N- to C-terminus in the order VH-CH1-CH2-CH3, referring to the heavy chain variable domain, heavy chain constant domain 1, heavy chain constant domain 2, and heavy chain constant domain 3 respectively. The IgG light chain is composed of two immunoglobulin domains linked from N- to C-terminus in the order VL-CL, referring to the light chain variable domain and the light chain constant domain respectively.

As used herein, the term “isotype” refers to the antibody class (e.g., IgG1, IgG2, IgG3, or IgG4 antibody) that is encoded by the heavy chain constant region genes. More particular the term “isotype” refers to IgG antibody classes.

The term “isolated” in the context of antibodies and antigen-binding fragments refers to an antibody or antigen-binding fragment thereof that is substantially free of other antibodies or antigen-binding fragments having different binding specificities. Moreover, an anti-TREM1 antibody or antigen-binding fragment may be substantially free of other cellular material and/or chemicals.

The term “effector molecule” as used herein includes, for example, antineoplastic agents, drugs, toxins, biologically active proteins, for example enzymes, other antibody or antigen-binding fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

As used herein “TREM1 polypeptide” or “TREM1 protein” refers to both wild-type sequences and naturally occurring variant sequences. TREM1 is a 234 amino acid immunoglobulin-like receptor membrane protein primarily expressed on myeloid lineage cells, including without limitation, macrophages, dendritic cells, monocytes, Langerhans cells of skin, Kupffer cells, osteoclasts, neutrophils and microglia. In some instances, TREM1 forms a receptor signaling complex with DAP12. In some instances, TREM1 may phosphorylate and signal through DAP12. Any fragment or variant of TREM1 are within the scope of the terms “TREM1 polypeptide” and “TREM1 protein”.

TREM1 proteins of the present invention include, without limitation, a mammalian TREM1 protein, human TREM1 protein (Uniprot Accession No. Q9NP99; SEQ ID NO: 1), mouse TREM1 protein (Uniprot Accession No. Q9JKE2; SEQ ID NO:2), rat TREM1 protein (Uniprot Accession No. D4ABU7; SEQ ID NO: 3), rhesus monkey TREM1 protein (Uniprot Accession No. F6TBB4; SEQ ID NO: 4).

The term “fragment” of a polynucleotide or polypeptide refers to any polynucleotide or polypeptide that differs from a reference polynucleotide or polypeptide sequence by being shorter than the reference sequence, such as by a terminal or internal. deletion. For example, a variant may be a result of alternative mRNA splicing. Alternative mRNA splicing can lead to tissue-specific patterns of gene expression by generating multiple forms of mRNA that can be translated into different protein products with distinct functions and regulatory properties.

The term “variant” or “variants” as used herein refers to polynucleotides or polypeptides that differ from a reference polynucleotide or polypeptide respectively. A variant and reference polypeptide may differ in amino acid sequence by one or more substitutions, additions, deletions, fusions and truncations, which may be present in any combination.

“Derivatives” or “variants” generally include those in which instead of the naturally occurring amino acid the amino acid which appears in the sequence is a structural analog thereof. In the context of antibodies, amino acids used in the sequences may also be derivatized or modified, e.g. labelled, providing the function of the antibody is not significantly adversely affected.

Derivatives and variants may be prepared during synthesis of the antibody or by post-production modification, or when the antibody is in recombinant form using the known techniques of site-directed mutagenesis, random mutagenesis, or enzymatic cleavage and/or ligation of nucleic acids.

The term “identity”, as used herein, indicates that at any particular position in the aligned sequences, the amino acid residue is identical between the sequences.

Degrees of identity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987, Sequence Analysis Primer, Gribskov, M. and Devereux, J., eds., M Stockton Press, New York, 1991, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402; Zhang, J. & Madden, T. L. 1997, Genome Res. 7:649-656).

An antibody “specifically binds” or “specifically recognizes” or “specific for” a protein when it binds with preferential or high affinity to the protein for which it is specific (or selective) but does not substantially bind, or binds with low affinity, to other proteins. The selectivity of an antibody may be further studied by determining whether or not the antibody binds to other related proteins as discussed above or whether it discriminates between them. Specific as employed herein is intended to refer to an antibody that only recognizes the antigen to which it is specific or an antibody that has significantly higher binding affinity to the antigen to which it is specific compared to binding to antigens to which it is non-specific, for example at least 5, 6, 7, 8, 9, 10 times higher binding affinity. Binding affinity may be measured by techniques such as BIAcore as described in WO2014/019727.

By specific (or selective), it will be understood that the antibody binds to the protein of interest with no significant cross-reactivity to any other molecule. Cross-reactivity may be assessed by any suitable method, such as BIAcore. Cross-reactivity of an antibody may be considered significant if the antibody binds to the other molecule at least about 50%, 10%, 150%, 20%, 25%, 30%, 35%, 40%, 45%, 50%, 55%, 60%, 65%, 70%, 75%, 80%, 85%, 90% or 100% as strongly as it binds to the protein of interest.

The term “modulating” in the context of antibodies refers to antibodies that bind their target antigen and modulate (e.g., decrease/inhibit or activate/induce) antigen function. For example, in case of TREM1, modulating antibodies modulate ligand binding to TREM1 and/or one or more TREM1 activities.

The term “neutralizing antibody” describes an antibody or an antigen-binding fragment thereof that is capable of inhibiting or attenuating the biological signaling activity of its target (target protein).

As used herein the term “blocking” in the context of the antibodies and antigen-binding fragments refers to antibodies and antigen-binding fragments that prevent other binders from binding to that antigen, such as, for example, occluding the receptor but will also include where the antibody or antigen-binding fragments thereof bind an epitope that causes, for example a conformational change which means that the natural ligand to the receptor no longer binds.

As used herein, “pharmaceutically acceptable carrier” includes any and all solvents, dispersion media, coatings, antibacterial and antifungal agents, isotonic and absorption delaying agents, and the like that are physiologically compatible. The carrier may be suitable for parenteral, e.g. intravenous, intramuscular, intradermal, intraocular, intraperitoneal, subcutaneous, spinal or other parenteral routes of administration, for example by injection or infusion. Alternatively, the carrier may be suitable for non-parenteral administration, such as a topical, epidermal or mucosal route of administration. The carrier may be suitable for oral administration. Depending on the route of administration, the modulator may be coated in a material to protect the compound from the action of acids and other natural conditions that may inactivate the compound.

“Pharmaceutically acceptable excipients” (vehicles, additives) are those inert substances that can reasonably be administered to a subject mammal and provide an effective dose of the active ingredient employed. These substances are added to a formulation to stabilize the physical, chemical and biological structure of the antibody. The term also refers to additives that may be needed to attain an isotonic formulation, suitable for the intended mode of administration.

A “subject,” “individual” or “patient” is used interchangeably herein, which refers to a vertebrate, preferably a mammal, more preferably a human. Mammals include, but are not limited to, murines, rats, simians, humans, farm animals, sport animals, and pets.

The term “motor neuron disease” as used herein, refers to diseases that primarily (but not necessarily exclusively) affect motor neurons, neuromuscular input or signal transmission at the neuromuscular junction. The motor neuron diseases referred above include, but are not limited to, amyotrophic lateral sclerosis (ALS), myasthenia gravis (MG), spinal muscular atrophy (SMA) or Charcot-Marie-Tooth disease (CMT).

The terms “prevent”, or “preventing” and the like, refer to obtaining a prophylactic effect in terms of completely or partially preventing a disease or symptom thereof. Preventing thus encompasses stopping the disease from occurring in a subject who may be predisposed to the disease but has not yet been diagnosed as having the disease.

The terms “treatment”, “treating” and the like, refer to obtaining a desired pharmacologic and/or physiologic effect. The effect may be therapeutic in terms of a partial or complete cure for a disease and/or adverse effect attributable to the disease. Treatment thus encompasses (a) inhibiting the disease, i.e., arresting its development; and (b) relieving the disease, i.e., causing regression of the disease.

In therapeutic applications, antibodies and antigen-binding fragments are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

The term “parenteral administration” as used herein means modes of administration other than enteral and topical administration, usually by injection.

As used herein “systemic administration” means administration into the circulatory system of the body (comprising the cardiovascular and lymphatic system), thus affecting the body as a whole rather than a specific locus such as the gastro-intestinal tract (via e.g., oral or rectal administration) and the respiratory system (via e.g., intranasal administration). Systemic administration can be performed e.g., by administering into muscle tissue (intramuscular), into the dermis (intradermal, transdermal, or supradermal), underneath the skin (subcutaneous), underneath the mucosa (submucosal), in the veins (intravenous) etc.

### Anti-TREM1 Antibodies and Antigen-Binding Fragments Thereof

The present invention demonstrates that antibodies and binding fragments thereof that bind and neutralize TREM1 can be used for the treatment of diseases in which microglia function is affected. Such function is important in motor neuron degenerative disorders, in particular, in ALS. Particular useful are antibodies and antigen-binding fragments thereof which inhibit one or more activities of TREM1. More specifically, antibodies and antigen-binding fragments prevent interaction of TREM1 with one or more of its natural ligands.

As described herein, the antibody for use in the present invention comprises a complete antibody molecule having full length heavy and light chains. Alternatively, the invention employs an antigen binding fragment.

Preferably said anti-TREM1 antibodies and antigen-binding fragment thereof is an isolated antibody and antigen-binding fragment thereof.

Antigen-binding fragments and methods of producing them are well known in the art, see for example Verma et al., 1998, Journal of Immunological Methods, 216, 165-181; Adair and Lawson, 2005. Therapeutic antibodies. Drug Design Reviews-Online 2(3):209-217. Examples of multi-specific antibodies or antigen-binding fragments thereof, which also are contemplated for use in the context of the disclosure, include bi, tri or tetra-valent antibodies, Bis-scFv, diabodies, triabodies, tetrabodies, bibodies and tribodies (see for example Holliger and Hudson, 2005, Nature Biotech 23(9): 1126-1136; Schoonjans et al. 2001, Biomolecular Engineering, 17(6), 193-202).

Antibodies generated against TREM1 polypeptide may be obtained, where immunization of an animal is necessary, by administering the polypeptides to an animal, preferably a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels or pigs may be immunized. However, mice, rabbits, pigs and rats are generally most suitable.

Antibodies for use in the invention may also be generated using single lymphocyte antibody methods by cloning and expressing immunoglobulin variable region cDNAs generated from single lymphocytes selected for the production of specific antibodies by, for example, the methods described by Babcook, J. et al., 1996, Proc. Natl. Acad. Sci. USA 93(15):7843-78481; WO92/02551; WO2004/051268 and International Patent Application number WO2004/106377.

More particular anti-TREM1 antibody is a monoclonal antibody. In a particular embodiment anti-TREM1 antibody or an antigen-binding fragment thereof is specific for TREM1.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

In one embodiment the antibody or fragments according to the disclosure are humanized. More particular the anti-TREM1 antibody thereof or antigen-binding fragment thereof is a human, humanized or chimeric antibody or antigen-binding fragment thereof

Suitably, the humanized antibody or antigen-binding fragment thereof according to the present invention has a variable domain comprising human acceptor framework regions as well as one or more of the CDRs and optionally further including one or more donor framework residues. Thus, provided in one embodiment is humanized antibody which binds to TREM1 wherein the variable domain comprises human acceptor framework regions and non-human donor CDRs.

When the CDRs or specificity determining residues are grafted, any appropriate acceptor variable region framework sequence may be used having regard to the class/type of the donor antibody from which the CDRs are derived, including mouse, primate and human framework regions.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody, fragment linked to an effector molecule, this may be prepared by standard chemical or recombinant DNA procedures in which the antigen-binding fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO 03/031581. Alternatively, where the effector molecule is a protein or polypeptide the linkage may be achieved using recombinant DNA procedures, for example as described in WO 86/01533 and EP0392745.

In an antibody or antigen-binding fragment thereof comprises a binding domain. A binding domain will generally comprise 6 CDRs, three from a heavy chain and three from a light chain. In one embodiment the CDRs are in a framework and together form a variable region. Thus in one embodiment an antibody or antigen-binding fragment thereof is a binding domain specific for TREM1 comprising a light chain variable region and a heavy chain variable region.

Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanized antibody of the present invention, the acceptor heavy and light chains do not necessarily need to be derived from the same antibody and may, if desired, comprise composite chains having framework regions derived from different chains.

More particular the anti-TREM1 antibody or antigen-binding fragment thereof comprises a human heavy chain constant region and a human light chain constant region.

More particular the anti-TREM1 antibody thereof is a full length antibody. More particular the anti-TREM1 antibody thereof is of the IgG isotype. More particular the anti-TREM1 antibody is selected from the group consisting of an IgG1, IgG4.

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required. It will be appreciated that sequence variants of these constant region domains may also be used. For example IgG4 molecules in which the serine at position 241 has been changed to proline as described in Angal et al., Molecular Immunology, 1993, 30 (1), 105-108 may be used. It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995). Accordingly, the C-terminal lysine of the antibody heavy chain may be absent.

In one embodiment, the anti-TREM1 antibody or antigen-binding fragment thereof binds to TREM1 with an affinity of at least 100 mM, 50 mM, 30 nM.

The affinity of an antibody or antigen-binding fragment thereof, as well as the extent to which an antibody or antigen-binding fragment thereof inhibits binding, can be determined by one of ordinary skill in the art using conventional techniques, for example those described by Scatchard et al. (Ann. KY. Acad. Sci. 51:660-672 (1949)) or by surface plasmon resonance (SPR) using systems such as BIAcore. For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al, Cancer Res. 53:2560-65 (1993)).

Antibodies and antigen-binding fragments of the present invention inhibit one or more TREM1 activities. Such inhibition results in the effects described in the examples, in particular, the effects on microglia function and migration and the levels of different markers. Antibodies and antigen-binding fragments thereof of the present invention may block TREM1 (blocking antibodies and antigen-binding fragments thereof) or otherwise interfere with TREM1 interactions with other proteins, such as its natural ligands such as peptidoglycan recognition protein 1 (PGLYRP1), high mobility group B1 (HMGB1), soluble CD177, heat shock protein 70 (HSP70). In a preferred embodiment, the anti-TREM1 antibody or antigen-binding fragment thereof blocks or prevents TREM1 interaction with PGLYRP1.

Disclosure herein relating to antibodies, particularly with respect to binding affinity and specificity, and activity, also is applicable to antigen-binding fragments and antibody-like molecules. It will be appreciated that antigen-binding fragments also may be characterized as monoclonal, chimeric, humanized, fully human, multi-specific, bi-specific etc., and that discussion of these terms also relate to antigen-binding fragments.

In one example the antibodies and antigen-binding fragments thereof bind to TREM1 as defined by SEQ ID NO: 1. Alternatively the antibody or antigen-binding fragment thereof binds to a TREM1 polypeptide as defined by SEQ ID NO: 1 or any variant or fragment thereof, more specifically any naturally-occurring variant or fragment thereof. In particular the antibody or antigen-binding fragment thereof binds to a polypeptide sequence which is at least 80% identical to the amino acid sequence of SEQ ID NO: 1.

The above antibodies and antigen-binding fragments thereof described for purposes of reference and example only and do not limit the scope of invention.

The anti-TREM1 antibodies or antigen-binding fragments thereof inhibiting TREM1 reduce the levels of costimulatory molecules (such as, for example, CD40, CD80, CD86) and activation markers (such as, for example, CD68, CSFR1). In a particular they reduce the levels of one or more of CD40, CD80, CD86, CD68, and CSFR1.

The antibody or antigen-binding fragment thereof also inhibits the migration of microglia. The migration of microglia can be measured using a scratch wound assay. Such assay is commonly used to measure cell migration.

As demonstrated by the present invention, the anti-TREM1 antibody or antigen-binding fragment thereof also shows reduction in the rate of phagocytosis in microglia.

In a particular embodiment the anti-TREM1 antibody or antigen-binding fragment thereof binds to an epitope on mouse TREM1 comprising one or more residues selected from 145, M46, K47, N50, Q71, R72, P73, T75, R76, P77, S78, S92, and E93, wherein the residue numbering is according to SEQ ID NO: 2.

Although these residues are provided for a particular sequence of mouse TREM1, the skilled person could readily extrapolate the positions of these residues to other corresponding TREM1 polypeptides (e.g. human or rat) using routine techniques. Antibodies binding to epitopes comprising the corresponding residues within these other TREM1 sequences are therefore also provided by the invention.

More specifically the present invention the anti-TREM1 antibody or antigen-binding fragment thereof binds to an epitope on human TREM1 comprising one or more residues selected from L45, E46, K47, S50, E71, R72, P73, K75, N76, S77, H78, D92, and H93, wherein the residue numbering is according to SEQ ID NO: 1.

In particular embodiment, the anti-TREM1 antibody or antigen-binding fragment thereof prevents interaction of TREM1 with PGLYRP1.

To screen for antibodies that bind to a particular epitope, a routine cross-blocking assay such as that described in Antibodies, Harlow and Lane (Cold Spring Harbor Press, Cold Spring Harb., NY) can be performed. Other methods include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Such methods are well known in the art.

Antibody epitopes may also be determined by X-ray crystallography analysis. Antibodies of the present invention may therefore be assessed through X-ray crystallography analysis of the antibody bound to TREM1

### In Vitro and Ex Vivo Use of Anti-TREM1 Antibodies and Antigen-Binding Fragments Thereof

The present invention provides an in vitro or ex vivo method of inhibiting phagocytic ability of microglia and/or migratory capacity of microglia, the method comprising contacting and incubating microglia cells with an antibody or antigen-binding fragment thereof that binds and neutralizes TREM1. More specifically, the anti-TREM1 antibody or antigen-binding fragment thereof prevents TREM1 interactions with one or more of its natural ligands. In a preferred embodiment, the anti-TREM1 antibody or antigen-binding fragment thereof prevents TREM1 interaction with PGLYRP1.

The cells are generally incubated for the time sufficient to allow anti-TREM1 antibody or an antigen-binding fragment thereof to bind to TREM1 and cause the biological effect.

The methods involving anti-TREM1 antibodies or an antigen-binding fragments thereof can be used to achieve biological effects as described in the Examples herein.

### Therapeutic Use of Anti-TREM1 Antibodies and Antigen-Binding Fragments Thereof

The present invention demonstrates the role of TREM1 is a key potentiator of microglia maladaptive neurotoxic responses in the context of neuron degenerative disorders such as ALS. Specifically, TREM1 inhibition reduces microglia neuronal uptake, pro-inflammatory cytokine release and microglia/peripheral immune migratory activities in vitro, ex vivo and in vivo models of ALS and attenuates brain and spinal cord inflammation in a SOD1G93A mouse model of ALS as described in the Examples herein. TREM1 inhibition in ALS can stop or attenuate disease progression by abrogating microglial, peripheral immune aberrant, and neurotoxic activation.

The present invention provides a method of treating or preventing a motor neuron degenerative disorder in a subject in need thereof, the method comprising administering to the subject an antibody or antigen-binding fragment thereof that binds and neutralizes TREM1. Such anti-TREM1 antibody or antigen-binding fragment thereof is administered in a therapeutically effective amount. In particular, such treatment or prevention is achieved by reducing microglia neuronal uptake and microglia migratory activities.

The present invention also provides an antibody or antigen-binding fragment thereof that binds and neutralizes TREM1 for use in the treatment of a motor neuron degenerative disorder.

In particular, as demonstrated by the Examples, the anti-TREM1 antibodies or antigen-binding fragment thereof attenuate brain and spinal cord inflammation in a subject diagnosed with a neuron degenerative disorder by reducing microglia neuronal uptake and microglia migratory activities.

Consequently the invention provides a method of attenuating brain and spinal cord inflammation in a subject diagnosed with a neuron degenerative disorder, the method comprising administering to said subject an antibody or antigen-binding fragment thereof that bind and neutralizes TREM1.

In yet another embodiment the present invention provides an antibody or antigen-binding fragment thereof that binds and neutralizes TREM1 for use in attenuating brain and spinal cord inflammation in a subject diagnosed with a neuron degenerative disorder.

In particular, such attenuation of brain and spinal cord inflammation is achieved by reducing microglia neuronal uptake and microglia migratory activities.

More specifically said motor neuron degenerative disorder is amyotrophic lateral sclerosis (ALS). In a specific embodiment ALS is characterized by a mutation in Superoxide dismutase 1 gene (SOD1).

### Pharmaceutical Compositions

An anti-TREM1 antibody or antigen-binding fragment thereof may be provided in a pharmaceutical composition. The pharmaceutical composition will normally be sterile and may additionally comprise a pharmaceutically acceptable adjuvant and/or carrier.

As the antibodies that bind and neutralize TREM1 are useful in the treatment and/or prophylaxis of a disorder or condition as described herein, the present invention also provides for a pharmaceutical composition comprising an antibody or antigen-binding fragment thereof that binds and neutralizes TREM1 in combination with one or more of a pharmaceutically acceptable carrier, excipient or diluent.

In particular the antibody or antigen-binding fragment thereof is provided as a pharmaceutical composition comprising one or more of a pharmaceutically acceptable excipient, diluent or carrier.

These compositions may comprise, in addition to the therapeutically active ingredient(s), a pharmaceutically acceptable excipient, carrier, diluent, buffer, stabilizer or other materials well known to those skilled in the art. Such materials should be non-toxic and should not interfere with the efficacy of the active ingredient.

Also provided are compositions, including pharmaceutical formulations, comprising an antibody, or polynucleotides comprising sequences encoding an antibody. In certain embodiments, compositions comprise one or more antibodies that bind and neutralize TREM1, or one or more polynucleotides comprising sequences encoding one or more antibodies that bind and neutralize TREM1. These compositions may further comprise suitable carriers, such as pharmaceutically acceptable excipients and/or adjuvants including buffers, which are well known in the art.

Pharmaceutical compositions of an antibody of the present invention are prepared by mixing such antibody having the desired degree of purity with one or more optional pharmaceutically acceptable carriers in the form of lyophilized formulations or aqueous solutions.

Examples of the techniques and protocols mentioned above can be found in Remington's Pharmaceutical Sciences, 20th Edition, 2000, pub. Lippincott, Williams & Wilkins.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be also prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The pharmaceutical compositions may include one or more pharmaceutically acceptable salts.

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Pharmaceutical compositions typically must be sterile and stable under the conditions of manufacture and storage. The composition can be formulated as a solution, microemulsion, liposome, or other ordered structure suitable to high drug concentration.

In one embodiment, the anti-TREM1 antibody is the sole active ingredient. In another embodiment, an anti-TREM1 antibody is in combination with one or more additional active ingredients. Alternatively, the pharmaceutical compositions comprise the antibody of the present invention which is the sole active ingredient and it may be administered individually to a patient in combination (e.g. simultaneously, sequentially or separately) with other agents, drugs or hormones.

The precise nature of the carrier or other material may depend on the route of administration, e.g. oral, intravenous, cutaneous or subcutaneous, nasal, intramuscular and intraperitoneal routes. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Solutions for intravenous administration or infusion may contain as carrier, for example, sterile water or preferably they may be in the form of sterile, aqueous, isotonic saline solutions.

Preferably, the pharmaceutical composition comprises a humanized antibody.

### Therapeutically Effective Amount and Dosage Determination

The anti-TREM1 antibodies and pharmaceutical compositions may be administered suitably to a patient to identify the therapeutically effective amount required. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. Compositions may be conveniently presented in unit dose forms containing a predetermined amount of an active agent of the disclosure per dose. Dose ranges and regimens for any of the embodiments described herein include, but are not limited to, dosages ranging from 1 mg-1000 mg unit doses.

A suitable dosage of an antibody or pharmaceutical composition may be determined by a skilled medical practitioner. Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.01 μg/kg to about 1000 mg/kg body weight, typically from about 0.1 μg/kg to about 100 mg/kg body weight, of the patient to be treated.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

### Administration of Pharmaceutical Compositions or Formulations

An antibody or pharmaceutical composition may be administered via one or more routes of administration using one or more of a variety of methods known in the art. As will be appreciated by the skilled person, the route and/or mode of administration will vary depending upon the desired results. Examples of routes of administration for the antibodies or pharmaceutical compositions include intravenous, intramuscular, intradermal, intraocular, intraperitoneal, subcutaneous, spinal or other parenteral routes of administration, for example by injection or infusion. Alternatively, the antibody or pharmaceutical composition may be administered via a non-parenteral route, such as a topical, epidermal or mucosal route of administration. The antibody or pharmaceutical composition may be for oral administration.

Suitable forms for administration include forms suitable for parenteral administration, e.g. by injection or infusion, for example by bolus injection or continuous infusion, in intravenous, inhalable or sub-cutaneous form. Where the product is for injection or infusion, it may take the form of a suspension, solution or emulsion in an oily or aqueous vehicle and it may contain additional agents, such as suspending, preservative, stabilizing and/or dispersing agents. Alternatively, the antibody or antigen-binding fragment thereof according to the invention may be in dry form, for reconstitution before use with an appropriate sterile liquid. Solid forms suitable for solution in, or suspension in, liquid vehicles prior to injection may also be prepared.

Preferably an antibody or antigen-binding fragment thereof that binds and neutralizes TREM1 is administered systemically. More specifically such antibody or antigen-binding fragment is administered subcutaneously or intravenously.

Once formulated, the pharmaceutical compositions can be administered directly to the subject.

### Articles of Manufacture and Kits

Kits comprising the antibodies and antigen-binding fragments thereof that bind and neutralize TREM1 and instructions for use are also provided. The kit may further contain one or more additional reagents, such as an additional therapeutic or prophylactic agent as discussed above.

In certain embodiments, the article of manufacture or kit comprises a container containing one or more of the antibodies of the invention, or the compositions described herein. In certain embodiments, the article of manufacture or kit comprises a container containing nucleic acids(s) encoding one (or more) of the antibodies or the compositions described herein. In some embodiments, the kit includes a cell of cell line that produces an antibody as described herein.

Accordingly, provided herein is the use of an antibody or an antigen-binding fragment thereof that binds and neutralizes TREM1 for the manufacture of a medicament for the treatment of motor neuron degenerative disorder.

The present invention also provides use of an antibody or antigen-binding fragment thereof that binds and neutralizes TREM1 for the manufacture of a medicament for attenuating brain and spinal cord inflammation in a subject diagnosed with a neuron degenerative disorder.

In certain embodiments, the article of manufacture or kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treatment, prevention and/or diagnosis and may have a sterile access port. At least one agent in the composition is an antibody of the present invention. The label or package insert indicates that the composition is used for the treatment of motor neuron degenerative disorder.

More specifically said motor neuron degenerative disorder is amyotrophic lateral sclerosis (ALS). In a specific embodiment ALS is characterized by a mutation in Superoxide dismutase 1 gene (SOD1).

It should be noted that the above-mentioned embodiments illustrate rather than limit the invention, and that those skilled in the art will be able to design many alternative embodiments without departing from the scope of the claims. In the claims, any reference signs placed between parentheses shall not be construed as limiting the claim.

### EXAMPLES

### Example 1. TREM1 Knockout Modulates Microglial Phagocytosis In Vitro

The effect of TREM1 knockout on the phagocytic ability of microglia was evaluated using a pH-sensitive fluorescent probe-conjugated zymosan phagocytosis assay. For isolation of primary microglia, forebrains were first isolated from post-natal day 7-8 TREM1−/− mice (Charles River) and B6NTac wild-type (WT) matching controls (Taconic). Meninges were carefully removed and brains were dissociated using the Papain Dissociation System (Worthington) according to the manufacturer's instructions. Homogenates were filtered through a 40 μm cell strainer (Falcon) and resuspended in complete medium. Single cell suspensions were then transferred into 175 flasks and incubated at 37° C. in 5% CO₂ for 7 days. Microglia were isolated from mixed glial cell cultures by shaking flasks for one hour at 200 rpm at 37° C., re-suspended in complete medium with 20 ng/ml of carrier-free macrophage colony stimulating factor (M-CSF; ThermoFisher) and grown for 7 days in 96-well (Greiner) plates at a density of 20,000 cells per well. Cells were then incubated for 30 mins with pHrodo®-conjugated zymosan bioparticles (12.5 μg/ml per well; ThermoFisher). Images were acquired during the assay using the InCell Analyzer 6000 system (GE Healthcare Life Sciences) with cell segmentation and particle counting performed using the InCellDeveloper Toolbox v1.9.

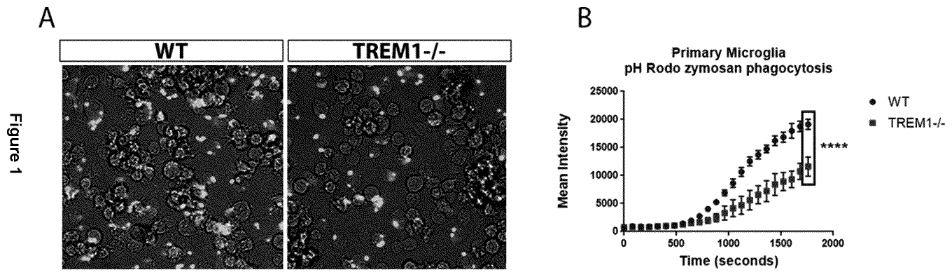

As shown in FIG. 1A uptake of zymosan particles was significantly reduced in TREM1−/− microglia relative to WT microglia.

### Example 2. TREM1 Knockout Reduces Migratory Capacity of Microglia In Vitro

The effect of TREM1 knockout on the migratory capacity of microglia was evaluated using a scratch wound migration assay. For isolation of primary microglia, forebrains were first isolated from post-natal day 7-8 TREM1−/− mice (Charles River) and B6NTac wild-type (WT) matching controls (Taconic). Meninges were carefully removed and brains were dissociated using the Papain Dissociation System (Worthington) according to the manufacturer's instructions. Homogenates were filtered through a 40 μm cell strainer (Falcon) and resuspended in complete medium. Single cell suspensions were then transferred into T75 flasks and incubated at 37° C. in 5% CO₂ for 7 days. Microglia were isolated from mixed glial cell cultures by shaking flasks for one hour at 200 rpm at 37° C., re-suspended in complete medium with 20 ng/ml of carrier-free macrophage colony stimulating factor (M-CSF; ThermoFisher) and grown for 7 days in 2-well culture insert 24-well (Ibidi) plates at a density of 30,000 cells/insert. Cells were incubated at 37° C. in 5% CO₂ until reaching approximately 80% confluence. Culture-inserts were then carefully removed followed by washing of the cell monolayer with fresh complete medium and imaging of the scratch area using an EVOS digital inverted light microscope. Extent of microglia cell migration into the scratch area was quantified using ImageJ.

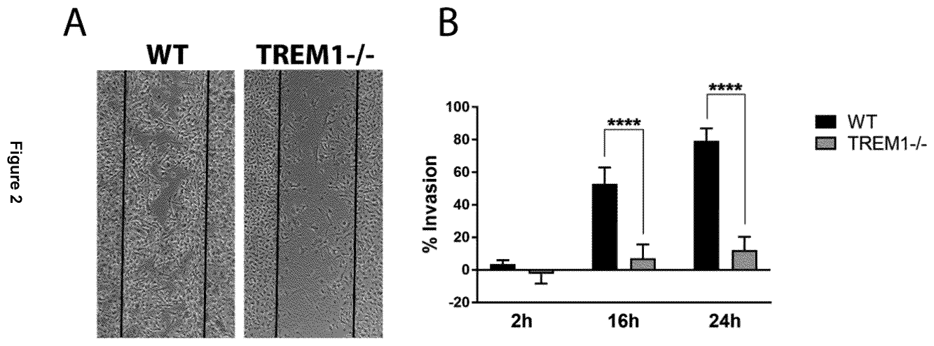

As shown in FIG. 2A, microglial migration into the wound area at 24 hours post-wound was significantly lower in TREM1−/− microglia relative to WT microglia.

### Example 3. TREM1 Knockout Decreases Levels of MCP-1 in LPS-Stimulated Microglia In Vitro

To evaluate the effects of TREM1 knockout on the ability of microglia to secrete chemotactic signals, levels of MCP-1 (CCL-2), a key chemokine that regulates migration and infiltration of monocytes/macrophages, were measured following lipopolysaccharide (LPS) stimulation. For isolation of primary microglia, forebrains were first isolated from post-natal day 7-8 TREM1−/− mice (Charles River) and B6NTac wild-type (WT) matching controls (Taconic). Meninges were carefully removed and brains were dissociated using the Papain Dissociation System (Worthington) according to the manufacturer's instructions. Homogenates were filtered through a 40 μm cell strainer (Falcon) and resuspended in complete medium. Single cell suspensions were then transferred into 175 flasks and incubated at 37° C. in 5% CO₂ for 7 days. Microglia were isolated from mixed glial cell cultures by shaking flasks for one hour at 200 rpm at 37° C., re-suspended in complete medium with 20 ng/ml of carrier-free macrophage colony stimulating factor (M-CSF; ThermoFisher) and grown for 7 days in 96-well (Greiner) plates at a density of 30,000 cells per well. Microglia were treated for 24 hours with 1 μg/ml of LPS from Escherichia coli (055:B5; Sigma-Aldrich) and supernatants collected for analysis of MCP-1 levels (MesoScale Discovery).

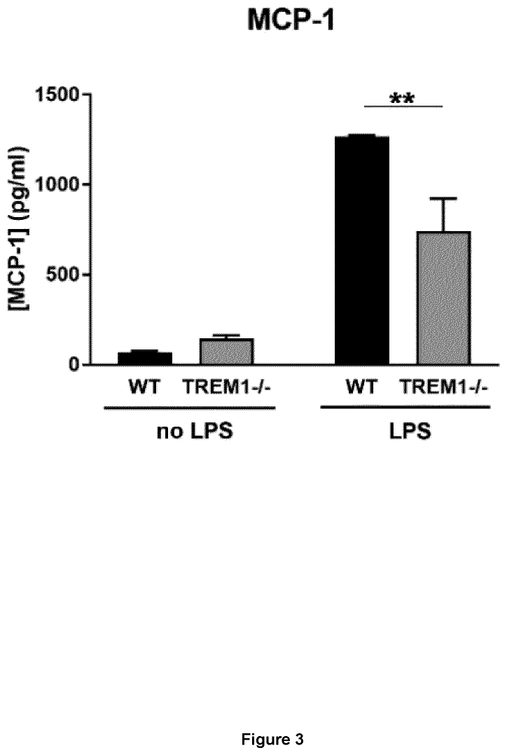

As shown in FIG. 3, following LPS stimulation, levels of MCP-1 were significantly lower in supernatants collected from TREM1−/− microglia relative to WT microglia.

### Example 4. Anti-TREM1 Antibody Reduces Migratory Capacity of Microglia In Vitro

The ability of a TREM1 antibody to modulate the migratory capacity of microglia was evaluated using a scratch wound assay. BV2 microglia cells were maintained in complete medium: DMEM GlutaMAX (MermoFisher) supplemented with 10% fetal bovine serum (FBS; ThermoFisher) and 1% penicillin/streptomycin (P/S; ThermoFisher) at 37° C. in 5% CO₂ in a humidified incubator. BV2 microglia were seeded at a density of 30,000 cells/insert in 2-well culture insert 24-well plates (Ibidi). Cells were incubated at 37° C. in 5% CO₂ until reaching approximately 80% confluence. Culture-inserts were then carefully removed followed by washing of the cell monolayer with fresh complete medium. Cells were then treated with isotype (IgG2A, MAB006, R&D Systems) or anti-mouse TREM1 (MAB1187, R&D Systems) antibody and the scratch area was imaged using an EVOS digital inverted light microscope. Extent of microglia cell migration into the scratch area was quantified using ImageJ.

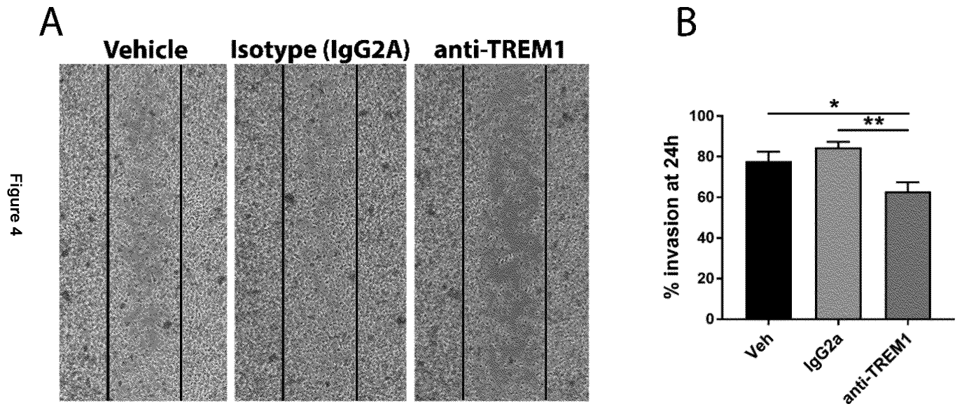

As shown in FIG. 4, BV2 migration into the wound area at 24 hours post-wound was lower in anti-TREM1 antibody treated microglia relative to isotype antibody- or vehicle-treated microglia.

### Example 5. TREM1 Activation Using Natural TREM1 Ligands Induces Release of Pro-Inflammatory Cytokines from Monocyte-Derived Macrophages

To assess the effects of TREM1 activation using proposed natural TREM1 ligands on the release of pro-inflammatory cytokines, human monocyte-derived macrophages (MDMs) were stimulated with peptidoglycan from Bacillus subtilis (PGN-BS) and peptidoglycan recognition protein 1 (PGLYRP1). To generate MDMs, monocytes were first isolated by leukopheresis from healthy human donors. Cells were resuspended in complete medium (DMEM Glutamax+10% FBS+1% P/S) supplemented with 40 ng/ml of carrier-free macrophage colony stimulating factor (M-CSF; Thermo Fisher) and cultured at a density of 5×10⁵ cells/ml in 24-well (Falcon) plates at 37° C. in 5% CO2 in a humidified incubator for 7 days. MDMs were then treated for 24 hours as follows: untreated control, PGN-BS (3 μg/ml; InvivoGen), PGLYRP1 (1 μg/ml; R&D Systems) and PGN-BS+PGLYRP1. Supernatants were then collected for analysis of TNF-α, IL-1β, IL-6 and IL-8 levels (MesoScale Discovery and R&D Systems Quantikine kits).

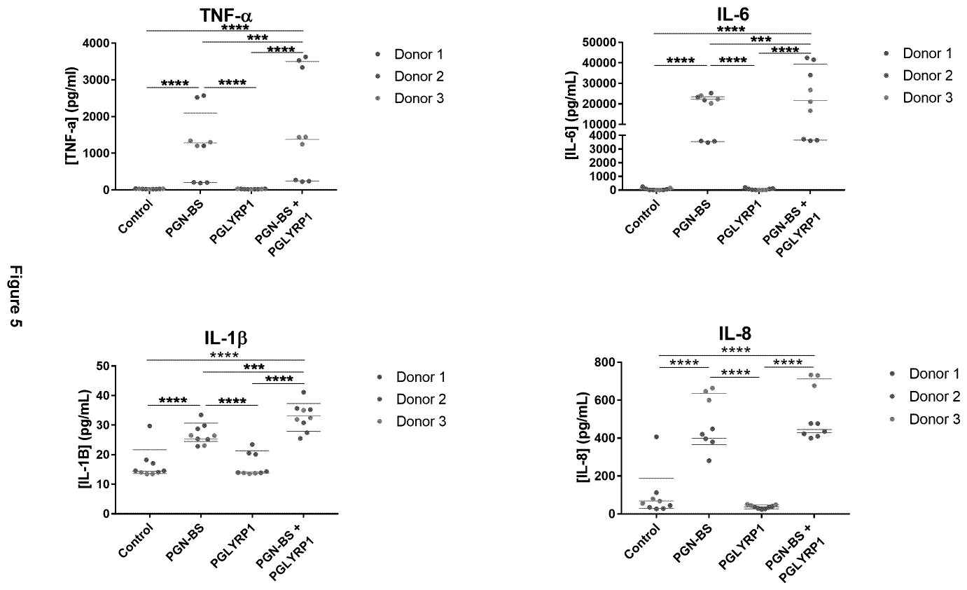

As shown in FIG. 5, treatment of MDMs with PGN-BS alone or PGN-BS+PGLYRP1 increased release of TNF-α, IL-1β, IL-6 and IL-8 from MDMs from 3 different donors relative to PGLYRP1 alone or untreated controls. For all 3 donors, IL-1β levels were higher following PGN-BS+PGLYRP1 treatment relative to PGN-BS alone. For 2 out of 3 donors, TNF-α and IL-6 levels were higher following PGN-BS+PGLYRP1 treatment relative to PGN-BS alone. IL-8 levels were not significantly different between PGN-BS and PGN-BS+PGLYRP1 treatments in all 3 donors.

### Example 6. TREM1 Knockout Modulates Microglial Phagocytosis Ex Vivo

The effect of TREM1 knockout on the phagocytic ability of microglia was measured using a pH-sensitive fluorescent probe-conjugated zymosan phagocytosis assay in ex vivo acute mouse brain slices. Brains from 5 TREM1−/− mice (Charles River) and 6 B6NTac wild-type (WT) matching controls (Taconic) were dissected and 300 μm thick sagittal sections were sliced using a vibratome VT1200S. Sections were allowed 1 h equilibrating in ice-cold artificial cerebrospinal fluid (A-CSF) choline buffer continuously bubbled with carbogen (95% O₂, 5% CO₂). They were then transferred in an incubator and incubated at 37° C. for another hour. 100 μl of pHrodog-conjugated zymosan bioparticles (ThermoFisher) were deposited on the top of the brain sections. After 1h incubation, brain sections were washed, fixed in 4% paraformaldehyde for 1 hour and immunostained by incubation with anti-Iba1 (microglial marker; Synaptic Systems) for 48 hours. Sections were then incubated with anti-rabbit Alexa-488-conjugated secondary antibody (ThermoFisher) for 3 hours and counterstained using DAPI (nucleus marker; ThermoFisher). Quantification of ex vivo microglial phagocytic activity and morphology was then performed based on a confocal LSM 880 (Zeiss) imaging followed by manual quantification of particle uptake. Microglial morphology was assessed through the use of a custom-made script using Fiji software.

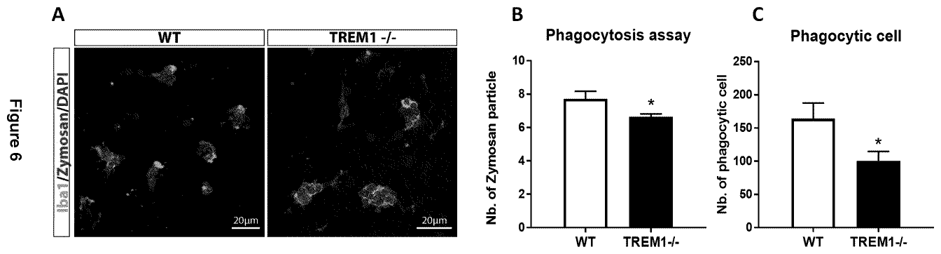

As shown in FIG. 6A, uptake of zymosan particles and the number of Iba1+ phagocytic cells was significantly reduced in TREM1−/− microglia relative to WT microglia.

### Example 7. TREM1 Knockout Reduces Synaptosome Uptake Ex Vivo

The effect of TREM1 knockout on the ability of microglia to phagocytose freshly isolated rat synaptosomes was assessed in ex vivo acute mouse brain slices. Brains were dissected from 3-month old Sprague-Dawley rats (Charles River), placed in 10 volumes of ice cold HEPES-buffered sucrose (0.32 M sucrose, 4 mM HEPES pH 7.4) and homogenized using a Dounce homogenizer. Homogenate was spun at 1000×g at 4° C. for 10 mins to remove the pelleted nuclear fraction (P1). The resulting supernatant was spun at 15,000×g for 20 mins to yield a crude synaptosomal pellet (P2) which was resuspended in 10 volumes of HEPES-buffered sucrose. After centrifugation at 10,000×g for an additional 15 mins, the washed crude synaptosomal fraction (P2′) was layered onto 4 ml of 1.2 M sucrose and centrifuged at 230,000×g for 15 mins. The interphase was collected, layered onto 4 ml of 0.8 M sucrose and centrifuged at 230,000×g (SW40 Ti rotor, Beckman Optima L-90K) for 15 mins to yield the synaptosome pellet. Purified synaptosomes were conjugated with pH-sensitive rhodamine-based pHrodo® Red succinimidyl ester (ThermoFisher, P36600) in 0.1 M sodium carbonate (pH 9.0) by incubation for 2 hrs at room temperature with gentle agitation. Unbound pHrodo® was removed by multiple rounds of washing and centrifugation with HBSS and pHrodo®-conjugated synaptosomes were then resuspended in HBSS with 5% DMSO and stored at −80° C. until use.

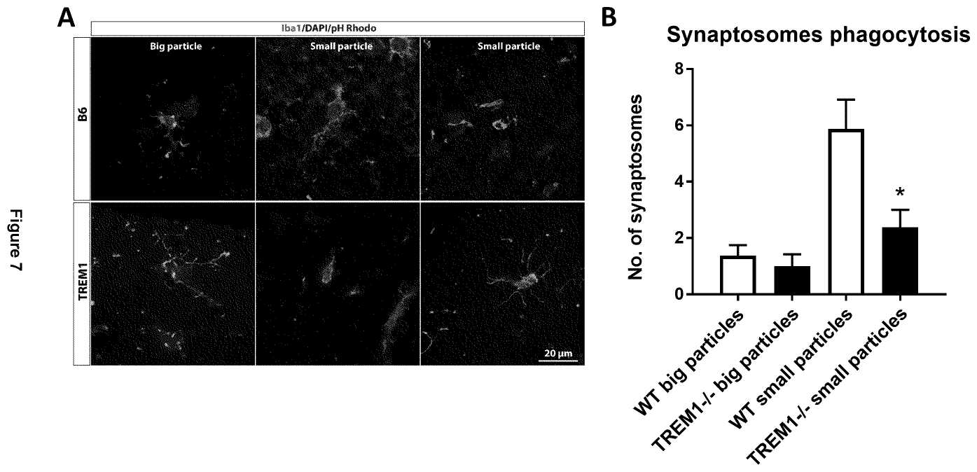

As shown in FIGS. 7A and 7B, uptake of synaptosomes was significantly reduced in the TREM1−/− microglia relative to WT microglia.

### Example 8. TREM1 Knock-Out Modulates Microglial Morphology Ex Vivo

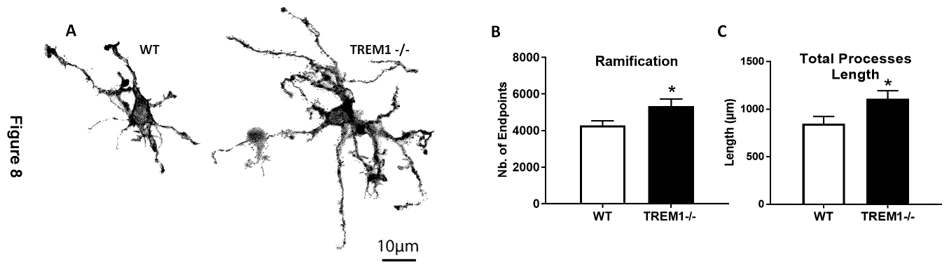

The effect of TREM1 knock-out on microglia morphology has been also evaluated. As shown in FIG. 8A, microglia from TREM1−/− mice also showed a striking change in their morphology. As shown in FIGS. 8B and 8C, this modification in morphology is reflected by significantly longer and more ramified processes in TREM1−/− microglia compared to WT controls.

### Example 9. Anti-TREM1 Antibody Reduces Spinal Cord Microglial Phagocytosis in SOD1-G93A Mice

The effect of a TREM1 antibody on the phagocytic ability of microglia in an ALS mouse model was evaluated using ex vivo acute spinal cord slices isolated from SOD1-G93A mice. SOD1-G93A mice (100 days of age; Jackson) were injected with either isotype (IgG2A, MAB006, R&D Systems) antibody or anti-mouse TREM1 (MAB1187, R&D Systems) antibody (two I.P. injections 48 hours apart). 24 hours after the second injection, the spinal cord was collected and immediately used for ex vivo slice generation. A segment of the spinal cord covering both thoracic and lumbar areas was selected, removed from meninges and immersed into a mold filled with low melting point agarose solution (Sigma). After solidification (1 min at 4° C.), 300 μm thick spinal cord sections were sliced using a vibratome VT1200S. Sections were allowed 1 hour equilibrating in ice-cold artificial cerebrospinal fluid (A-CSF) choline buffer continuously bubbled with carbogen (95% O₂, 5% CO₂). They were then transferred in an incubator and incubated at 37° C. for another hour. 100 μl of pHrodo®-conjugated zymosan bioparticles (MermoFisher) were deposited on the top of the spinal cord sections. After one hour incubation, spinal cord sections were washed, fixed in 4% paraformaldehyde for 1 hour and immunostained by incubation with anti-Iba1 (microglial marker; Synaptic Systems) for 48 hours. Sections were then incubated with anti-rabbit Alexa-488-conjugated secondary antibody (MermoFisher) for 3 hours and counterstained using DAPI (nucleus marker; ThermoFisher). Quantification of ex vivo microglial phagocytic activity and morphology was then performed based on a confocal LSM 880 (Zeiss) imaging followed by manual quantification of particle uptake.

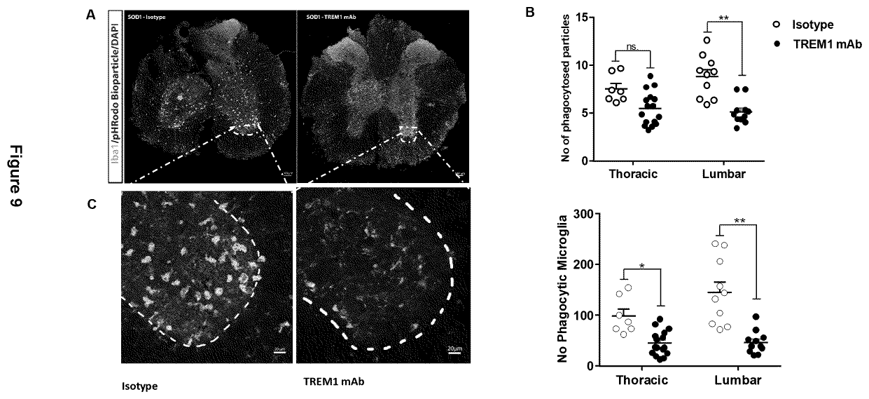

As shown in FIG. 9A, anti-TREM1-treated SOD1-G93A mice showed decreased microgliosis compared to isotype-treated SOD1-G93A controls. As shown in FIG. 9B, microglia from anti-TREM1-treated SOD1-G93A mice displayed reduced phagocytic uptake (microglial efficiency) compared to isotype-treated controls. There were also reduced total numbers of phagocytic microglia (microglial abundance) in anti-TREM1-treated SOD1-G93A mice. FIG. 9C shows the ventral horn region from the images in 9A.

### Example 10. Inhibition of TREM1 Reduces Levels of Co-Stimulatory Molecules and Activation Markers in SOD1-G93A Mice

The effects of a TREM1 antibody on brain inflammation in SOD1-G93A mice were assessed using a mass cytometry approach. SOD1-G93A mice (100 days of age) were injected with either isotype (IgG2A, MAB006, R&D Systems) antibody or anti-mouse TREM1 (MAB1187, R&D Systems) antibody (two I.P. injections 48 hours apart). 24 hours after the second injection, mice were anaesthetized and perfused with 1×HBSS (10 U/ml heparin) for 5 mins. Forebrains were collected in ice-cold 1×HBSS and dissociated using the Papain Dissociation System (Worthington) according to the manufacturer's instructions. Single cell suspensions were filtered, resuspended in 30% Percoll in HBSS and centrifuged for 15 mins at 500×g with no brake to remove myelin. The cell pellet was washed with Maxpar cell staining buffer (Fluidigm) and then stained with a cocktail of rare metal-tagged antibodies (Fluidigm, markers listed below) for 1 hour (100 μl final staining volume per sample). Cells were washed 3 times in Maxpar cell staining buffer and fixed in 4% PFA in PBS (prepared from 16% formaldehyde, ThermoFisher). Maxpar DNA Intercalator (50 nM, Fluidigm) was incubated with the cells overnight at 4° C. to identify live/dead cells. After washing twice in PBS, cells were washed in Maxpar H₂O (Fluidigm) and centrifuged for 5 mins at 800×g. Cells were then re-suspended in Maxpar H₂O, and metal isotope bead standards (EQ Four Element Calibration Beads, Fluidigm) added to the sample for data normalization. Single cell suspensions were analyzed on a CyTOF Helios mass cytometer (Fluidigm) with events acquired at approximately 500 events per second. Data were analyzed using Cytobank software (Cytobank Inc.).

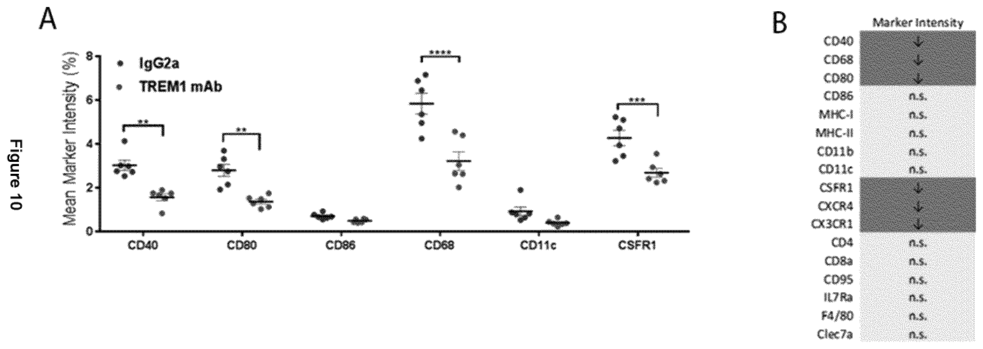

As shown in FIG. 10A, treatment of SOD1-G93A mice with a TREM1 antibody reduced levels of costimulatory molecules (CD40, CD80, CD86) and other activation markers (CD68, CSFR1) compared to isotype-treated SOD1-G93A controls As shown in FIG. 10B, a number of costimulatory molecules and other activation markers were significantly reduced in anti-TREM1-treated SOD1-G93A mice compared to isotype-treated SOD1-G93A controls (arrows represent significant changes in anti-TREM1-treated SOD1-G93A mice).

### Example 11. Brain Penetration of Anti-TREM1 Antibody in SOD1-G93A Mice

The extent of brain penetration of an anti-TREM1 antibody in SOD1-G93A mice was assessed using a mass cytometry approach. SOD1-G93A mice (100 days of age) were injected with either a biotinylated isotype (IgG2A, IC006B, R&D Systems) antibody or biotinylated anti-mouse TREM1 (BAM1187, R&D Systems) antibody (two I.P. injections 48 hours apart). 24 hours after the second injection, mice were anaesthetized and perfused with 1×HBSS (10 U/ml heparin) for 5 mins. Forebrains and spleens were collected in ice-cold 1×HBSS. Brains were dissociated using the Papain Dissociation System (Worthington) according to the manufacturer's instructions. Single cell suspensions were filtered, resuspended in 30% Percoll in HBSS and centrifuged for 15 mins at 500×g with no brake to remove myelin. Spleens were mechanically homogenized, filtered and red blood cell contaminants removed using red blood cell lysis buffer (ThermoFisher). Cell pellets were washed with Maxpar cell staining buffer (Fluidigm) and then stained with anti-biotin (1D4-C5, Fluidigm) for 1 hour (100 μl final staining volume per sample). Cells were washed 3 times in Maxpar cell staining buffer and fixed in 4% PFA in PBS (prepared from 16% formaldehyde, ThermoFisher). Maxpar DNA Intercalator (50 nM, Fluidigm) was incubated with the cells overnight at 4° C. to identify live/dead cells. After washing twice in PBS, cells were washed in Maxpar H₂O (Fluidigm) and centrifuged for 5 mins at 800×g. Cells were then re-suspended in Maxpar H₂O, and metal isotope bead standards (EQ Four Element Calibration Beads, Fluidigm) added to the sample for data normalization. Single cell suspensions were analyzed on a CyTOF Helios mass cytometer (Fluidigm) with events acquired at approximately 500 events per second. Data were analyzed using Cytobank software (Cytobank Inc.).

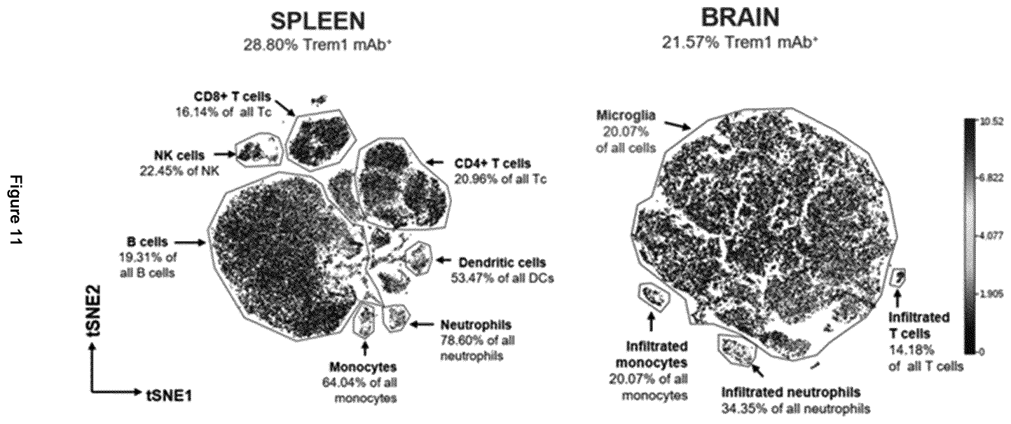

As shown in FIG. 11, tSNE averaged data showed that 21.57% and 28.80% of all immune cells in the brain and spleen respectively were positive for the anti-TREM1 antibody in anti-TREM1-treated SOD1-G93A mice.

### Example 12. Determination of the Epitope of MAB1187 and its Equivalent in Human TREM1

An array of 37 mouse TREM1 IgV domain (positions 21-136 of SEQ ID NO: 2) mutant clones has been produced. Each of the clones had 2 surface residues, in close proximity, mutated to alanine and was fused to a human Fc. In addition to the mutant clones the Wild Type clone was also included. Sequences of the mutant mouse TREM1 array clones including the wild type are shown in Table 1.

Each of the above clones is expressed as an Fc fusion protein and captured on a sensor coated with an anti-human Fc antibody. This fusion protein consisted of TREM1 IgV domains followed by a triple alanine linker fused to a human Fc domain ensuring that TREM1 will be presented in a bivalent format Subsequently, the sensors are dipped in an antibody solution and the binding kinetics are monitored using a Bio-Layer Interferometry (BLI) instrument (octet RED384, ForteBio)

Once all the mutant TREM1 Fc clones have been loaded on sensor tips (38 tips used per run), the sensors are dipped in a solution containing the antibody for which the epitope needs identification. By monitoring the binding kinetics of the antibody to each of these mutants and comparing them to the kinetics against the wild type protein we can deduct the epitope. A decrease in the ab dissociation constant for a clone indicates that the mutated residues are important for antibody binding and hence are part of its epitope.

The above mouse TREM1 array was loaded on 38 anti-human Fc sensors and was used to monitor the kinetics of the R+D monoclonal antibody MAB1187. The results are shown in Table 2.

Using the above method the epitope has been determined as follows: residues 145, M46, K47, N50, Q71, R72, P73, T75, R76, P77, S78, S92, and E93 (the positions correspond to SEQ ID NO: 2).

Mouse and human TREM1 sequences have been aligned using Clustal omega (pyMol can also be used, which does a structural alignment). The following corresponding epitope residues in human TREM1 have been identified (the positions correspond to SEQ ID NO: 1): L45, E46, K47, S50, E71, R72, P73, K75, N76, S77, H78, D92, and H93.

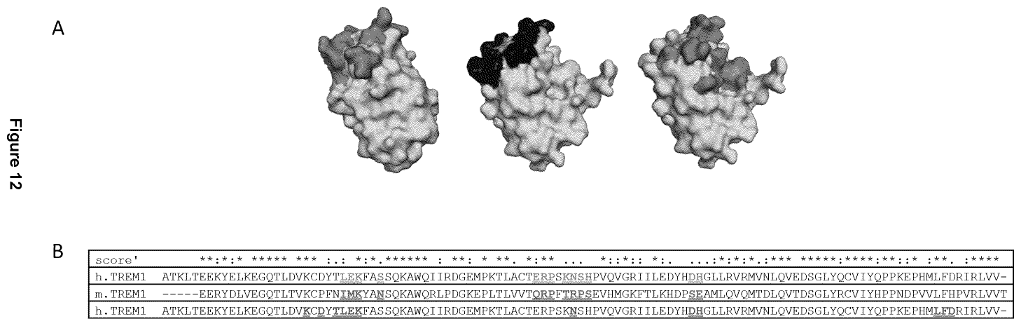

FIG. 12A shows a 3D representation of mouse and human TREM1 with the MAB1187 epitope on the mouse TREM1 structure and the human TREM1. The PGLYRP1 epitope on the human TREM1 is also shown (structure on the right). The sequence alignment of human and mouse TREM1 with the epitope of MAB1187 and PGLYRP1 indicates that MAB1187 binds to TREM1 in a manner preventing it from binding to PGLYRP1 ligand.

TABLE 1
Mutant clones of mouse TREM1 and wild
type sequence.
Ala mutation are highlighted.

| | |
|---|---|
| WT | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 1 | AAALEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 2 | AIAAEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 3 | AIVAAEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 4 | AIVLEAARYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 5 | AIVLEEAAYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 6 | AIVLEEERYALVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLAVTKG |
| 7 | AIVLEEERYDLAEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVAKG |
| 8 | AIVLEEERYDLVEGAALTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 9 | AIVLEEERYDLVEGQTLAVKCPFNIMKYANSQKAWQRLPD |
| | GKEPLTLVVTQRPFTRPSEVHMGKFTLKHDPSEAMLQVAM |
| | TDLQVTDSGLYRCVIYHPPNDPVVLFHPVRLVVTKG |
| 10 | AIVLEEERYDLVEGQTLTVACAFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 11 | AIVLEEERYDLVEGQTLTVKCPFAAMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 12 | AIVLEEERYDLVEGQTLTVKCPFNAAKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 13 | AIVLEEERYDLVEGQTLTVKCPFNIAAYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 14 | AIVLEEERYDLVEGQTLTVKCPFNIMKYAASQKAWQRLPD |
| | GKEPLTLVVTQRPFARPSEVHMGKFTLKHDPSEAMLQVQM |
| | TDLQVTDSGLYRCVIYHPPNDPVVLFHPVRLVVTKG |
| 15 | AIVLEEERYDLVEGQTLTVKCPFNIMKYANSQKAWQRLPA |
| | GAEPLTLVVTQRPFTRPSEVHMGKFTLKHDPSEAMLQVQM |
| | TDLQVTDSGLYRCVIYHPPNDPVVLFHPVRLVVTKG |
| 16 | AIVLEEERYDLVEGQTLTVKCPFNIMKYANSQKAWQRLPD |
| | GAAPLTLVVTQRPFTRPSEVHMGKFTLKHDPSEAMLQVQM |
| | TDLQVTDSGLYRCVIYHPPNDPVVLFHPVRLVVTKG |
| 17 | AIVLEEERYDLVEGQTLTVKCPFNIMKYANSQKAWQRLPD |
| | GKAPATLVVTQRPFTRPSEVHMGKFTLKHDPSEAMLQVQM |
| | TDLQVTDSGLYRCVIYHPPNDPVVLFHPVRLVVTKG |
| 18 | AIVLEEERYDLVEGQTLTVKCPFNIMKYANSQKAWQRLPD |
| | GKEPLTLVVTAAPFTRPSEVHMGKFTLKHDPSEAMLQVQM |
| | TDLQVTDSGLYRCVIYHPPNDPVVLFHPVRLVVTKG |
| 19 | AIVLEEERYDLVEGQTLTVKCPFNIMKYANSQKAWQRLPD |
| | GKEPLTLVVTQAAFTRPSEVHMGKFTLKHDPSEAMLQVQM |
| | TDLQVTDSGLYRCVIYHPPNDPVVLFHPVRLVVTKG |
| 20 | AIVLEEERYDLVEGQTLTVKCPFNIMKYANSQKAWQRLPD |
| | GKEPLTLVVTQRPFAAPSEVHMGKFTLKHDPSEAMLQVQM |
| | TDLQVTDSGLYRCVIYHPPNDPVVLFHPVRLVVTKG |
| 21 | AIVLEEERYDLVEGQTLTVKCPFNIMKYANSQKAWQRLPD |
| | GKEPLTLVVTQRPFTAASEVHMGKFTLKHDPSEAMLQVQM |
| | TDLQVTDSGLYRCVIYHPPNDPVVLFHPVRLVVTKG |
| 22 | AIVLEEERYDLVEGQTLTVKCPFNIMKYANSQKAWQRLPD |
| | GKEPLTLVVTQRPFTRAAEVHMGKFTLKHDPSEAMLQVQM |
| | TDLQVTDSGLYRCVIYHPPNDPVVLFHPVRLVVTKG |
| 23 | AIVLEEERYDLVEGQTLTVKCPFNIMKYANSQKAWQRLPD |
| | GKEPLTLVVTQRPFTRPAAVHMGKFTLKHDPSEAMLQVQM |
| | TDLQVTDSGLYRCVIYHPPNDPVVLFHPVRLVVTKG |
| 24 | AIVLEEERYDLVEGQTLTVKCPFNIMKYANSQKAWQRLPD |
| | GKEPLTLVVTQRPFTRPSEVAAGKFTLKHDPSEAMLQVQM |
| | TDLQVTDSGLYRCVIYHPPNDPVVLFHPVRLVVTKG |
| 25 | AIVLEEERYDLVEGQTLTVKCPFNIMKYANSQKAWQRLPD |
| | GKEPLTLVVTQRPFTRPSAVHMGKFTLAHDPSEAMLQVQM |
| | TDLQVTDSGLYRCVIYHPPNDPVVLFHPVRLVVTKG |
| 26 | AIVLEEERYDLVEGQTLTVKCPFNIMKYANSQKAWQRLPD |
| | GKEPLTLVVTQRPFTRASEVHMGKFTLKHAPSEAMLQVQM |
| | TDLQVTDSGLYRCVIYHPPNDPVVLFHPVRLVVTKG |
| 27 | AIVLEEERYDLVEGQTLTVKCPFNIMKYANSQKAWQRLPD |
| | GKEPLTLVVTQRPFTRPSEVHMGKFTLKHDPAAAMLQVQM |
| | TDLQVTDSGLYRCVIYHPPNDPVVLFHPVRLVVTKG |
| 28 | AIVLEEERYDLVEGQTLTVKCPFNIMKYANSQKAWQRLPD |
| | GKEPLTLVVTQRPFTRPSEVHMGKFTLKHDPSEAMLQVQM |
| | AALQVTDSGLYRCVIYHPPNDPVVLFHPVRLVVTKG |
| 29 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLAATDSGLYRCVIYHPPNDPVVLFHPVRLVVTKG |
| 30 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQAADSGLYRCVIYHPPNDPVVLFHPVRLVVTKG |
| 31 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYAAPNDPVVLFHPVRLVVTKG |
| 32 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHAANDPVVLFHPVRLVVTKG |
| 33 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPAAPVVLFHPVRLVVTKG |
| 34 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPAALFHPVRLVVTKG |
| 35 | AIVLEAERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFAPVRLVVTKG |
| 36 | AIVLEEEAYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVALVVTKG |
| 37 | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVAAG |

TABLE 2
The sensors that showed reduced dissociation
constants. Ala mutations are highlighted
within the sequences and the corresponding
mutations on the wild type sequence are
underlined.

| | |
|---|---|
| WT | AIVLEEERYDLVEGQTLTVKCPFNIMKYANSQKAWQRLPD |
| | GKEPLTLVVTQRPFTRPSEVHMGKFTLKHDPSEAMLQVQM |
| | TDLOVTDSGLYRCVIYHPPNDPVVLFHPVRLVVTKG |
| 12 | AIVLEEERYDLVEGQTLTVKCPFNAAKYANSQKAWQRLPD |
| | GKEPLTLVVTQRPFTRPSEVHMGKFTLKHDPSEAMLOVOM |
| | TDLQVTDSGLYRCVIYHPPNDPVVLFHPVRLVVTKG |
| 13 | AIVLEEERYDLVEGQTLTVKCPFNIAAYANSQKAWQRLPD |
| | GKEPLTLVVTQRPFTRPSEVHMGKFTLKHDPSEAMLQVQM |
| | TDLQVTDSGLYRCVIYHPPNDPVVLFHPVRLVVTKG |
| 14 | AIVLEEERYDLVEGQTLTVKCPFNIMKYAASQKAWQRLPD |
| | GKEPLTLVVTQRPFARPSEVHMGKFTLKHDPSEAMLOVOM |
| | TDLQVTDSGLYRCVIYHPPNDPVVLFHPVRLVVTKG |
| 18 | AIVLEEERYDLVEGQTLTVKCPFNIMKYANSQKAWQRLPD |
| | GKEPLTLVVTAAPFTRPSEVHMGKFTLKHDPSEAMLQVQM |
| | TDLQVTDSGLYRCVIYHPPNDPVVLFHPVRLVVTKG |
| 19 | AIVLEEERYDLVEGQTLTVKCPFNIMKYANSQKAWQRLPD |
| | GKEPLTLVVTQAAFTRPSEVHMGKFTLKHDPSEAMLQVQM |
| | TDLQVTDSGLYRCVIYHPPNDPVVLFHPVRLVVTKG |
| 20 | AIVLEEERYDLVEGQTLTVKCPFNIMKYANSQKAWORLPD |
| | GKEPLTLVVTQRPFAAPSEVHMGKFTLKHDPSEAMLQVQM |
| | TDLQVTDSGLYRCVIYHPPNDPVVLFHPVRLVVTKG |
| 21 | AIVLEEERYDLVEGQTLTVKCPFNIMKYANSQKAWQRLPD |
| | GKEPLTLVVTQRPFTAASEVHMGKFTLKHDPSEAMLQVQM |
| | TDLQVTDSGLYRCVIYHPPNDPVVLFHPVRLVVTKG |
| 22 | AIVLEEERYDLVEGQTLTVKCPFNIMKYANSQKAWORLPD |
| | GKEPLTLVVTQRPFTRAAEVHMGKFTLKHDPSEAMLQVQM |
| | TDLQVTDSGLYRCVIYHPPNDPVVLFHPVRLVVTKG |
| 27 | AIVLEEERYDLVEGQTLTVKCPFNIMKYANSQKAWQRLPD |
| | GKEPLTLVVTQRPFTRPSEVHMGKFTLKHDPAAAMLQVQM |
| | TDLQVTDSGLYRCVIYHPPNDPVVLFHPVRLVVTKG |

### Example 13. Binding Kinetics of MAB1187

The kinetics of Mab1187 binding to mouse TREM1 were measured at 25° C. by surface plasmon resonance on a Biacore T200 instrument.

A goat anti rat IgG, Fc fragment specific antibody (F(ab)′2 fragment, Jackson ImmunoResearch 112-005-071) was immobilized on a CM5 Sensor Chip via amine coupling chemistry to a level of approximately 10000 RU. A reference cell was treated with the same amine coupling chemistry, but was not brought into contact with the antibody. After amine coupling was complete, all subsequent solutions were flowed over the reference cell and the sample cell in series, and the response of the reference cell was subtracted from the sample cell throughout the run.

Each analysis cycle consisted of capture of approximately 250 RU of MAB1187 to the anti Fc surface, injection of analyte for 180 s (at 25° C. at a flow rate of 30 μl per minute), dissociation of analyte for 600 s, followed by surface regeneration (with a 60 s injection of 50 mM HCl, a 30 s injection of 5 mM NaOH, and a further 60 s injection of 50 mM HCl). Mouse TREM-1 analyte (in house, his tagged) was injected at 3-fold serial dilutions in HBS-EP+ running buffer (GE Healthcare) at concentrations of 300 nM to 3.7 nM. Buffer blank injections were included to subtract instrument noise and drift.

Kinetic parameters were determined using a 1:1 binding model using Biacore 1200 Evaluation software (version 3.0). The fitting parameters of RI (representing bulk shift) and Rmax (representing signal of a fully bound complex) were both set to use a local fit. MAB1187 was shown to have an affinity of 26 nM for mouse TREM1. The kinetic parameters are summarized in Table 3.

TABLE 3
Kinetic parameters of MAB1187 binding to mouse TREM1.

| kₐ (1/Ms) | k_{d} (1/s) | K_{D} (nM) | n= |
|---|---|---|---|
| 2.0E+05 | 5.1E−03 | 26 | 1 |

### Example 14. Brain Penetration of Anti-TREM1 Antibody in SOD1-G93A Mice

The extent of brain penetration of an anti-TREM1 antibody in SOD1-G93A mice was also assessed quantifying the antibody by liquid chromatography with mass spectrometry detection (LCMS/MS). SOD1-G93A mice (15 weeks of age) and their non-transgenic littermate controls were injected intraperitoneally with an anti-TREM1 antibody (#MAB1187, clone 174031, R&D systems) at 30 mg/kg (n=3 animals/genotype). Blood for plasma isolation was collected from the lateral tail vein in microvette tubes containing clotting activator (CB 300, 16.440, Sarstedt) at 48h after the antibody injection. Serum was obtained by allowing the blood to coagulate at room temperature for 30-60 minutes and subsequent centrifugation at 2000 g for 10 minutes at 4° C. The supernatant was collected, slowly frozen on dry ice and stored at −80° C. until further use. Afterwards, animals were anesthetized with 0.1 ml of undiluted pentobarbital (Dolethal, Vetoquinol) and transcardially perfused with HBSS supplemented with 0.2% heparin for 5 minutes at 6 ml/min. Spinal cord and brain hemispheres were rapidly dissected, snap-frozen in liquid nitrogen and stored at −80° C. until further use.

Samples were prepared for analysis using a total lysis assay. First brain and spinal cord samples were diluted 2 fold in PBS buffer and then mixed with Precellys homogenizer (Bertin Instruments) instruments 2 times during 30 sec at 4500 rpm. 25 μL of each sample were aliquoted in micronic tubes, as well as calibration standards, quality control samples and blanks. Then 30 μL of internal standard working solution, prepared by diluting the stock solution in 33/67 H2O/ACN, were added to each tube. Samples were consequently denaturated using 7 μL of TCEP and incubated for 30 minutes at room temperature. Afterwards samples were alkylated using 7 μL of iodoacetamide and incubated for 30 minutes at room temperature and protected from light. 170 μL of a mix constituted per well of 7 μL of L-cysteine, 153 μL of ammonium bicarbonate pH 7.9 buffer and 10 μL of trypsin solution at 0.5 mg/mL in acetic acid were added to each tube. Samples were incubated overnight for 16 to 21 hours at 37° C. Samples were then centrifuged for 5 minutes at approximately 1500 g. The trypsinization reaction was stopped by transferring 100 μL of supernatant to a 96-well plate containing 100 μL of 92% H2O 5% MeOH 3% formic acid. The plate was analysed by two dimension LC-MS/MS. The instrument was an ultra-performance liquid chromatography from Shimadzu coupled to a triple quadrupole mass spectrometer from Sciex (6500+ system). For the first dimension LC, stationary phase used was a BEH C4 column of 2.1×100 mm dimensions from Waters and mobile phases used were Bicarbonate buffer 10 mM/MEOH 95/5 and Bicarbonate buffer 10 mM/MEOH 5/95. For the second dimension LC, stationary phase used was a BEH C18 column of 2.1×100 mm dimensions from Waters and mobile phases used are H2O+0.1% propionic acid and ACN+0.1% formic acid. The MS instrument was used in MRM mode and following transitions were used to monitor the two peptides of interest: 615,330->654,382 and 421,9->513,3 respectively for the signature peptide and the internal standard. Data processing was performed on Analyst software (Sciex).

There was not notable differences between SOD1-G93A mice and wild type mice in the exposure levels of antibody in serum, brain or spinal cord. The averaged concentration on anti-TREM1 antibody 48 after intraperitoneal administration of 30 mg/kg observed in SOD1-G93A mice was 259 μg/mL, 0.826 g/g and 0.874 μg/g in serum, brain and spinal cord, respectively, and in type mice was 277 μg/mL, 0.998 μg/g and 1.114 μg/g in serum, brain and spinal cord, respectively. The brain-to-serum concentration ratios of anti-TREM1 antibody observed 48h after intraperitoneal administration of 30 mg/kg was 0.33% and 0.38% in SOD1-G93A and wild type mice, respectively. The spinal cord-to-serum concentration ratios of anti-TREM1 antibody observed 48h after intraperitoneal administration of 30 mg/kg was 0.42% and 0.35% in SOD1-G93A and wild type mice, respectively. The brain-to-spinal cord concentration ratios of anti-TREM1 antibody observed 48h after intraperitoneal administration of 30 mg/kg was 0.85% and 0.95% in SOD1-G93A and wild type mice, respectively suggesting similar exposure to anti-TREM1 antibody in brain and spinal cord. These data suggest that the CNS exposure to the antibody was around 0.3% of the levels in serum and that there was not differences in exposure between SOD1-G93A and wild type mice. The brain-to-serum ratios and spinal cord-to serum ratios are similar to values reported in rodent with other antibodies.

All references cited herein, including patents, patent applications, papers, textbooks and the like, and the references cited therein, to the extent that they are not already, are hereby incorporated herein by reference in their entirety.

### REFERENCES

Al-Chalabi and Hardiman, (2013) Nat Rev Neurol. 9(11):617-28
Al-Chalabi et al., (2017) Nat Rev Neurol. 13(2):96-104
Beers, D. R. et. al. (2019) Lancet Neurol. 18(2):211-220
Boille, S. et. al. (2006) Science 312: 1389-1391
Buchon, A et al. (2000) J. Immunol. 164:4991-4995
Butovsky et al (2012) J Clin Inv 122(9):3063-308
Colona, M. (2003) Nat. Immunol. Rev. 3: 1-9
Harms, M. et al. (2014) 82 (10 supplement) Neurology
Lincencum et al (2010) Nat Genetics 42(5):392-9
Philips et al (2015) Curr Protoc Pharmacol. 1M 69
Turner, M. et al. (2013) Neurology. 81(14): 1222-1225

What is claimed is:

1. A method of treating a motor neuron degenerative disorder in a subject in need thereof, the method comprising systemically administering to the subject an antibody or antigen-binding fragment thereof that binds and neutralizes TREM1.

2. An antibody or antigen-binding fragment thereof that binds and neutralizes TREM1 for use in the treatment of a motor neuron degenerative disorder, wherein said antibody or antigen-binding fragment thereof is administered systemically.

3. Use of an antibody or antigen-binding fragment thereof that binds and neutralizes TREM1 for the manufacture of a medicament for systemic administration for the treatment of a motor neuron degenerative disorder.

4. The method, the antibody or antigen-binding fragment thereof or the use according to any one of claims 1 to 3, wherein the antibody or antigen-binding fragment thereof prevents TREM1 interactions with one or more of its natural ligands.

5. The method, the antibody or antigen-binding fragment thereof or the use according to claim 4, wherein said natural ligand is Peptidoglycan Recognition Protein 1 (PGLYRP1).

6. The method, the antibody or antigen-binding fragment thereof or the use according to any one of claims 1 to 4, wherein said motor neuron degenerative disorder is amyotrophic lateral sclerosis (ALS).

7. The method, the antibody or antigen-binding fragment thereof or the use according to claim 5, wherein ALS is characterized by the presence of a mutation in SOD1 gene.

8. The method, the antibody or antigen-binding fragment thereof or the use according to any claims 1 to 4, wherein the antibody or antigen-binding fragment thereof is administered subcutaneously or intravenously.

9. The method, the antibody or antigen-binding fragment thereof, or the use according to any one of claims 1 to 4, wherein said antibody or antigen-binding fragment thereof binds to TREM1 with an affinity of at least 50 nM.

10. The method, the antibody or antigen-binding fragment thereof or the use according to any one of claims 1 to 4, wherein said treatment reduces microglia neuronal uptake.

11. The method, the antibody or antigen-binding fragment thereof or the use according to any one of claims 1 to 4, wherein said treatment inhibits the migration of microglia.

12. The method, the antibody or antigen-binding fragment thereof or the use according to claim 11, wherein the migration is measured using a scratch wound assay.

13. The method, the antibody or antigen-binding fragment thereof or the use according to any one of claims 1 to 4, wherein said antibody or antigen-binding fragment thereof reduces the rate of phagocytosis in microglia.

14. The method, the antibody or antigen-binding fragment thereof or the use according to any one of claims 1 to 4, wherein said antibody or antigen binding fragment thereof is a monoclonal antibody or antigen-binding fragment thereof.

15. The method, the antibody or antigen-binding fragment thereof or the use according to any one of claims 1 to 4, wherein said antibody or antigen-binding fragment is a human, humanized or chimeric antibody or antigen-binding fragment thereof.

16. The method, the antibody or the use according to any one of claims 1 to 4, wherein the antibody is a full length antibody.

17. The method, the antibody or antigen-binding fragment thereof or the use according to any one of claims 1 to 4, wherein said antibody or antigen-binding fragment comprises a human heavy chain constant region and a human light chain constant region.

18. The method, the antibody or the use according to any one of claims 1 to 4, wherein said antibody is of the IgG isotype.

19. The method, the antibody or the use according to any one of claims 1 to 4, wherein the antibody is an IgG1 or IgG4.

20. The method, the antibody or antigen-binding fragment thereof or the use according to any claims 1 to 19, wherein the anti-TREM1 antibody or antigen-binding fragment is provided as a pharmaceutical composition comprising one or more of a pharmaceutically acceptable excipient, diluent or carrier.

21. An in vitro or ex vivo method of inhibiting phagocytic ability of microglia and/or migratory capacity of microglia, the method comprising contacting and incubating microglia cells with an antibody or antigen-binding fragment thereof that binds and neutralizes TREM1.


SEQUENCE LISTING (42 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 230 aa, Mus musculus
MRKAGLWGLLCVFFVSEVKAAIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKGSSDVFTPVIIPITRLTERPILITTKYSPSDTTTTRSLPKPTAVVSSPGLGVTIINGTDADSVSTSSVTISVICGLLSKSLVFIILFIVTKRTFG

SEQ ID NO: 3 - protein, 226 aa, Rattus norvegicus
MRKAGLWGLLLVFFVSEVKAAIVPEEERYDLVEGQTLTVNCPFNIMKYARSRKAWQRLSAGKEPLTLVVTERSSTTSSEVRVGKYTLKDDPTEAMLFVQMTDLQVTDSGLYRCVIYHPPNDPVLLFHPVRLVVTKGSSGVSVPDIIPTTKPTEVPVLITTKHSTPTRSLPKSTAVVSSPDPGVTINNGTDPTSVSTYNVVVPVVCGLLIKTLIFFVLFVVTKRSFG

SEQ ID NO: 4 - protein, 201 aa, Macaca mulatta
MRKTRLWGLLWMLFVSELRATTELTEEKYEYKEGQTLEVKCDYALEKYANSRKAWQKMEGKMPKILAKTERPSENSHPVQVGRITLEDYPDHGLLQVQMTNLQVEDSGLYQCVIYQHPKESHVLFNPICLVVTKGSSGTPGSSENSTQNVYRTPSTTAKALGPRYTSPRTVTQAPPESTVVVSTPGPGPLPFFPSPCAERM

SEQ ID NO: 5 - protein, 116 aa, Mus musculus
AIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 6 - protein, 116 aa, Artificial Sequence
  Mutant protein
AAALEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 7 - protein, 116 aa, Artificial Sequence
  Mutant sequence
AIAAEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 8 - protein, 116 aa, Artificial Sequence
  Mutant sequence
AIVAAEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 9 - protein, 116 aa, Artificial Sequence
  Mutant sequence
AIVLEAARYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 10 - protein, 116 aa, Artificial Sequence
  Mutant sequence
AIVLEEAAYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 11 - protein, 116 aa, Artificial Sequence
  Mutant sequence
AIVLEEERYALVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLAVTKG

SEQ ID NO: 12 - protein, 116 aa, Artificial Sequence
  Mutant sequence
AIVLEEERYDLAEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVAKG

SEQ ID NO: 13 - protein, 116 aa, Artificial Sequence
  Mutant sequence
AIVLEEERYDLVEGAALTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 14 - protein, 116 aa, Artificial Sequence
  Mutant sequence
AIVLEEERYDLVEGQTLAVKCPFNIMKYANSQKAWQRLPDGKEPLTLVVTQRPFTRPSEVHMGKFTLKHDPSEAMLQVAMTDLQVTDSGLYRCVIYHPPNDPVVLFHPVRLVVTKG

SEQ ID NO: 15 - protein, 116 aa, Artificial Sequence
  Mutant sequence
AIVLEEERYDLVEGQTLTVACAFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 16 - protein, 116 aa, Artificial Sequence
  Mutant sequence
AIVLEEERYDLVEGQTLTVKCPFAAMKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 17 - protein, 116 aa, Artificial Sequence
  Mutant sequence
AIVLEEERYDLVEGQTLTVKCPFNAAKYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 18 - protein, 116 aa, Artificial Sequence
  Mutant sequence
AIVLEEERYDLVEGQTLTVKCPFNIAAYANSQKAWQRLPDGKEPLTLVVTQRPFTRPSEVHMGKFTLKHDPSEAMLQVQMTDLQVTDSGLYRCVIYHPPNDPVVLFHPVRLVVTKG

SEQ ID NO: 19 - protein, 116 aa, Artificial Sequence
  Mutant sequence
AIVLEEERYDLVEGQTLTVKCPFNIMKYAASQKAWQRLPDGKEPLTLVVTQRPFARPSEVHMGKFTLKHDPSEAMLQVQMTDLQVTDSGLYRCVIYHPPNDPVVLFHPVRLVVTKG

SEQ ID NO: 20 - protein, 116 aa, Artificial Sequence
  Mutant sequence
AIVLEEERYDLVEGQTLTVKCPFNIMKYANSQKAWQRLPAGAEPLTLVVTQRPFTRPSEVHMGKFTLKHDPSEAMLQVQMTDLQVTDSGLYRCVIYHPPNDPVVLFHPVRLVVTKG

SEQ ID NO: 21 - protein, 116 aa, Artificial Sequence
  Mutant sequence
AIVLEEERYDLVEGQTLTVKCPFNIMKYANSQKAWQRLPDGAAPLTLVVTQRPFTRPSEVHMGKFTLKHDPSEAMLQVQMTDLQVTDSGLYRCVIYHPPNDPVVLFHPVRLVVTKG

SEQ ID NO: 22 - protein, 116 aa, Artificial Sequence
  Mutant sequence
AIVLEEERYDLVEGQTLTVKCPFNIMKYANSQKAWQRLPDGKAPATLVVTQRPFTRPSEVHMGKFTLKHDPSEAMLQVQMTDLQVTDSGLYRCVIYHPPNDPVVLFHPVRLVVTKG

SEQ ID NO: 23 - protein, 116 aa, Artificial Sequence
  Mutant sequence
AIVLEEERYDLVEGQTLTVKCPFNIMKYANSQKAWQRLPDGKEPLTLVVTAAPFTRPSEVHMGKFTLKHDPSEAMLQVQMTDLQVTDSGLYRCVIYHPPNDPVVLFHPVRLVVTKG

SEQ ID NO: 24 - protein, 116 aa, Artificial Sequence
  Mutant sequence
AIVLEEERYDLVEGQTLTVKCPFNIMKYANSQKAWQRLPDGKEPLTLVVTQAAFTRPSEVHMGKFTLKHDPSEAMLQVQMTDLQVTDSGLYRCVIYHPPNDPVVLFHPVRLVVTKG

SEQ ID NO: 25 - protein, 116 aa, Artificial Sequence
  Mutant sequence
AIVLEEERYDLVEGQTLTVKCPFNIMKYANSQKAWQRLPDGKEPLTLVVTQRPFAAPSEVHMGKFTLKHDPSEAMLQVQMTDLQVTDSGLYRCVIYHPPNDPVVLFHPVRLVVTKG

SEQ ID NO: 26 - protein, 116 aa, Artificial Sequence
  Mutant sequence
AIVLEEERYDLVEGQTLTVKCPFNIMKYANSQKAWQRLPDGKEPLTLVVTQRPFTAASEVHMGKFTLKHDPSEAMLQVQMTDLQVTDSGLYRCVIYHPPNDPVVLFHPVRLVVTKG

SEQ ID NO: 27 - protein, 116 aa, Artificial Sequence
  Mutant sequence
AIVLEEERYDLVEGQTLTVKCPFNIMKYANSQKAWQRLPDGKEPLTLVVTQRPFTRAAEVHMGKFTLKHDPSEAMLQVQMTDLQVTDSGLYRCVIYHPPNDPVVLFHPVRLVVTKG

SEQ ID NO: 28 - protein, 116 aa, Artificial Sequence
  Mutant sequence
AIVLEEERYDLVEGQTLTVKCPFNIMKYANSQKAWQRLPDGKEPLTLVVTQRPFTRPAAVHMGKFTLKHDPSEAMLQVQMTDLQVTDSGLYRCVIYHPPNDPVVLFHPVRLVVTKG

SEQ ID NO: 29 - protein, 116 aa, Artificial Sequence
  Mutant sequence
AIVLEEERYDLVEGQTLTVKCPFNIMKYANSQKAWQRLPDGKEPLTLVVTQRPFTRPSEVAAGKFTLKHDPSEAMLQVQMTDLQVTDSGLYRCVIYHPPNDPVVLFHPVRLVVTKG

SEQ ID NO: 30 - protein, 116 aa, Artificial Sequence
  Mutant sequence
AIVLEEERYDLVEGQTLTVKCPFNIMKYANSQKAWQRLPDGKEPLTLVVTQRPFTRPSAVHMGKFTLAHDPSEAMLQVQMTDLQVTDSGLYRCVIYHPPNDPVVLFHPVRLVVTKG

SEQ ID NO: 31 - protein, 116 aa, Artificial Sequence
  Mutant sequence
AIVLEEERYDLVEGQTLTVKCPFNIMKYANSQKAWQRLPDGKEPLTLVVTQRPFTRASEVHMGKFTLKHAPSEAMLQVQMTDLQVTDSGLYRCVIYHPPNDPVVLFHPVRLVVTKG

SEQ ID NO: 32 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPAAAMLQVQMTDLQVTDSGLYRCVIYHPPNDPVVLFHPVRLVVTKG

SEQ ID NO: 33 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMAALQVTDSGLYRCVIYHPPNDPVVLFHPVRLVVTKG

SEQ ID NO: 34 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLAATDSGLYRCVIYHPPNDPVVLFHPVRLVVTKG

SEQ ID NO: 35 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLQAADSGLYRCVIYHPPNDPVVLFHPVRLVVTKG

SEQ ID NO: 36 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYAAPNDPVVLFHPVRLVVTKG

SEQ ID NO: 37 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHAANDPVVLFHPVRLVVTKG

SEQ ID NO: 38 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPAAPVVLFHPVRLVVTKG

SEQ ID NO: 39 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPAALFHPVRLVVTKG

SEQ ID NO: 40 - protein, 116 aa, Artificial Sequence
  Mutant sequence
AIVLEAERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFAPVRLVVTKG

SEQ ID NO: 41 - protein, 116 aa, Artificial Sequence
  Mutant sequence
AIVLEEEAYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVALVVTKG

SEQ ID NO: 42 - protein, 116 aa, Artificial Sequence
  Mutant sequence
AIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVAAG